(19) United States
(12) Patent Application Publication
(10) Pub. No.: US 2003/0194430 A1
(43) Pub. Date: Oct. 16, 2003

(54) Process for encapsulating multi-phase, multi-compartment capsules for therapeutic compositions

(76) Inventors: Frederick H. Miller, Tampa, FL (US); Lance R. Ausec, St. Pete Beach, FL (US)

Correspondence Address: PATE PIERCE & BAIRD, 215 SOUTH STATE STREET, SUITE 550, PARKSIDE TOWER, SALT LAKE CITY, UT, 84111, US

(21) Appl. No.: 10/369,247

(22) Filed: Feb. 18, 2003

Related U.S. Application Data
(60) Provisional application No. 60/371,448, filed on Apr. 10, 2002.

(51) Int. Cl. A61K 9/48; B31B 1/60; B29C 39/10

(52) U.S. Cl. 424/451; 156/60; 264/4

(57) ABSTRACT

An encapsulation process for forming a multi-compartment capsule. The process comprising the steps of: (1) providing a primary capsule having a base and a cap; (2) providing a secondary capsule having a base and a cap; (3) introducing at least one ingredient having a first physical state (i.e., solid, liquid, gas or dispersion) into a receiving chamber within the internal periphery of the secondary capsule; (4) positioning the cap of the secondary capsule into a sealing relationship with the base of the secondary capsule; (5) introducing at least one ingredient having a second physical state (i.e., solid, liquid, gas or dispersion) into a receiving chamber within the internal periphery of the primary capsule; (6) introducing the secondary capsule into the internal periphery of the primary capsule; and positioning the cap of the primary capsule into a sealing relationship with the base of the primary capsule. The ingredients introduced within the primary and secondary capsules comprise at least one active ingredient or medicament (e.g., pharmaceutical, biotechnical, nutraceutical, vitamin, dietary supplement, mineral or combination thereof). The component parts of the multi-compartment capsule may include various time-release coatings to facilitate release of active ingredient(s) or medicaments at different rates. The capsular cap may be configured in such a manner or a filling material may be introduced into the cap to substantially reduce any potential dead space volume within the capsule, thereby minimizing the opportunity for reaction between an air bubble and one or more of the active ingredients introduced into the capsule.

### RELATED APPLICATIONS

This application claims the benefit of U.S. Provisional Application Serial No. 60/371,448, filed Apr. 10, 2002, and entitled “INTEGRATED CAPSULE DELIVERY APPARATUS AND METHOD,” which is hereby incorporated herein by reference.

### BACKGROUND

1. The Field of the Invention

The present invention relates to delivery of active ingredients or medicaments and, more particularly, to novel capsular delivery apparatus and methods for delivering one or more active ingredients or medicaments having diverse physical states (e.g., solid, liquid, gas or dispersion) into a single dosage, multi-compartment capsule.

2. Background of the Invention

As appreciated by those skilled in the art, the contemplation, design, testing and manufacture of chemicals and biomolecules for administration to humans and animals, as nutritional or therapeutic agents, requires a thorough integration of clinically contemplated delivery principles and modalities. Chemicals and biomolecules that may be administered to humans and animals are often referred to herein as “actives,” “active ingredients” or “medicaments.”

Oral administration has become one of the most frequent routes for delivering one or more active ingredients or medicaments to the body. Active ingredients or medicaments, such as nutritional or therapeutic agents, may be orally administered in a variety of physical states (i.e., solid, liquid or gas). Tablets and capsules are generally the most common vehicle for the oral delivery of medicaments. As appreciated, a tablet may be broadly characterized as a compressed powder or granular solid. Prior to compression of the granular powder comprising the medicament into tablet form, the presence of one or more excipients may be required. An excipient includes any inert substance (i.e., gum arabic, starch or the like) combined with a principal ingredient to facilitate the preparation of an agreeable or convenient dosage form of the active or medicament. Functional characteristics of excipients may include, for example, disintegration, lubrication, appearance, palatability, shelf-stability or the like.

Those skilled in the art also developed capsule as a contrivance for containing a solid or liquid dosage form of a medicament. Traditional capsular embodiments include a first containment section referred to as a base, and a second containment section referred to as a cap. The two pieces of the capsule are usually formulated and designed in a manner such that the material to be encapsulated may be introduced into the base section, whereas the open end of the cap section may be correspondingly positioned over the open end of the base. The walls of the cap and base are generally in physical contact with one another to form a single internal cavity. A means for structurally sealing the cap in relation to the base may also be incorporated during manufacture to insure non-tampering of the capsule. In this regard, those skilled in the art developed sealing technology which contemplates banding, heat fusion (spot-welding) and snap seals which utilize a “tongue and groove” scheme.

The outer walls of a capsule are preferably formed of a soluble ingredient, such as, for example, gelatin (animal-based product), starch, hydrophillic polymer or hydroxypropyl methyl-cellulose (HPMC), which provides a barrier for containing the active ingredient or medicament, in powder or liquid form, within the internal periphery of the capsule walls. Traditionally, hard gelatin capsules may be manufactured by dipping plates of stainless steel pins into a pool of gelatin solution. The pins are then removed from the gelatin and rotated while the gelatin is dried in a kiln with forced, humidity-controlled air. Once dried, the gelatin capsules are typically stripped from the pins, trimmed to a suitable length and then joined together (e.g., base and cap) and packaged for production use.

With the advent of automated encapsulation machinery, the responsibility to produce encapsulated products shifted mainly to industrial manufacturers. Contemporaneous with the development of the encapsulation industry, those skilled in the art have advanced the state of the encapsulation art. For example, several significant improvements in encapsulation technology have been seen over the last forty years. These technological improvements have included, for example, the development of soft elastic capsules, film-coating techniques, micro-encapsulation and multiple-compartment technology.

Soft elastic capsules, often referred to as soft gelatin capsules, were developed in an effort to provide means for encapsulating liquids and other medicaments which are typically poorly soluble in water. In preferred design, soft elastic capsules are made from a thicker and more plastic gelatin having an increased flexibility due to the addition of a polyol, such as glycerin or sorbitol. The addition of such plasticizers has been found, however, to have the potential disadvantage of increasing the risk for microbial growth. Thus an antimicrobial, such as a paraben or sorbic acid, may be added to the soft elastic capsule shell in order to address any microbial concern.

Prior art film-coating techniques generally involve a plating process, whereby a thin, uniform film may be deposited onto the outer surface of the delivery vehicle (e.g., tablet or capsule). Several successive layers may be deposited onto the outer surface of the vehicle, if desired, in an effort to facilitate various desirable properties. For example, sugar-coating, a precursor to film-coating, has been used by those skilled in the art for more than one hundred years to make tablets more palatable. Other advantages or properties of film-coating may include for example, but not by way of limitation, protection from moisture, oxidation, controlling microbial contamination and inhibiting modification of the chemical properties of the active ingredient. As further appreciated by those skilled in the art, prior art film-coating may form an interfacial barrier between two chemicals or chemical compounds that might otherwise react when they come into contact.

Enteric coatings and sustained-release formulations are contemplated as variations on prior art film-coating techniques. In particular, enteric coating describes a process where the delivery vehicle (e.g., tablet or capsule) is coated with one or more layers of chemicals that are somewhat resistant to extreme pH conditions. For example, conditions of extremely low pH are commonly encounter in the stomach. Many active ingredients or medicaments are in the form of a pharmaceutical salt and thus highly susceptible to ionization in the presence of hydrogen ions. Thus, the presence of an enteric coating generally provides a level of protection as to degradation of the active ingredient or medicament until transit from the stomach into the small intestine is accomplished.

Film coatings have also led to the development of delivery vehicles (e.g., tablets and capsules) having sustained-release properties. Mixtures of waxes, cellulose, silicone and similar resins have been found useful by those skilled in the art for creating-sustained release coatings. In principle, these prior art coatings function to delay the release of the active ingredient or medicament to the targeted body system, thereby facilitating a timed, absorption rate in the body. Furthermore, the entire daily dosage of an active or medicament may be contained in a single, sustained-release delivery vehicle (e.g., tablet or capsule), whereas the immediate absorption of the entire dosage could possibly lead to an overdosage of the medicament. Thus, by layering quanta of medicament with differential coatings, the dosage undergoes a controlled release over specified time period. The application of sustained-release film coating technology therefore may inherently facilitate the delivery of a total daily dosage amount of an active or medicament to be released to the body in controlled increments.

Over the last several years, a considerable amount of attention has been focused on the further development of multi-compartment capsule technology for the delivery of therapeutic and diagnostic agents. Series formulations teach the use of membranes or other types of barriers to cordon a line of separate chambers within a single encapsulating shell. As appreciated, the purpose of such multi-compartment delivery devices is the administration of multiple dosages. Moreover, multiple-compartment delivery mechanisms of the prior art were developed to circumvent or diminish the effects of harsh pH environments within humans. For example, the prior art contemplates a hard capsule formulation which contains three different compartments of active medicaments for administration to the vaginal and rectal areas. In preferred structure, the formulation outer, rapid-release layer may contain an active medicament and excipient; the middle, intermediate-release layer may include a powder form of active medicament; and the inner, slow-release layer may contain pellets or granules of active medicament.

Also taught in the prior art are multi-compartment capsules having groups of spheroids with pH-dependent coatings which are encapsulated within a hard gelatin shell and provided for treating female yeast infection. The first spheroid is preferably uncoated and may be in a powder form; the second spheroid may contain a pH sensitive coat; and the inner spheroid may include a pH insensitive coat.

In addition to pH-sensitive coatings, hydrogels and other gastric retention technologies have been developed by those skilled in the art in an effort to retard the progression of the delivery vehicle during enteric transit. This retarding action, presumably, allows the full amount of active medicament to be released and/or targeted to a specific area of the gastrointestinal tract. Hydrogel and related gastric retention devices of the prior art generally rely upon the imbibing of water into a center core which is filled with cellulose or similar water absorbent material. In preferred operation, the material swells and releases multiple compartments of active medicament. The concept of using bulk size to slow transit of single active medicament in a single physical state is thus appreciated.

In an effort to administer active ingredients or medicaments to a specific location in the body to treat a specific disorder caused by a specific pathogen, those skilled in the art have used targeted-release systems using multi-compartment capsular technology. For example, a method for carrying out a triple therapy against the microorganisms Helicobacter pylori, a known infectious agent which is believed largely responsible for the development of gastric ulcer disease, was developed which comprises the steps of oral administration of a pharmaceutical dosage form comprising an internal capsule placed inside an external capsule, wherein the external capsule comprises a soluble salt of bismuth and a first antibiotic, and the internal capsule comprises a second antibiotic. In addition, multi-compartmental capsules were developed which combine a nutrient supplement with a viable direct-fed microbial (i.e., gastrointestinal microorganisms, including bacteria, live cell yeasts, fungi or a combination thereof) for the purpose of treating livestock for feeding disorders and improving feed efficiency.

A disadvantage with prior art encapsulation technology is when the base and corresponding cap of a capsule are joined, dead space volume is typically created within the internal periphery of the capsule. Internal capsular dead space may be filed with an air bubble which may ultimately react with one or more of the active ingredients or medicaments introduced within the capsule, thereby potentially degrading the quality and effectiveness of the active ingredients.

Although the prior art discloses multiple compartment, capsular delivery technology, these manifestations generally includes one of two approaches. For example, one approach contemplates the introduction of a single active or medicament into multiple capsular compartments to vary the temporal release of the medicament and ultimately the absorption rate into the body. Another approach contemplates the introduction of a plurality of active ingredients or medicaments into different compartments of a single capsule for delivery to a specific area of the body to treat a targeted illness or condition.

The use or contemplation of multiple-compartment capsular delivery apparatus or methods which deliver different physical forms of the same active or medicament, or a variation in physical forms of different actives or medicaments in a single dosage, however, has not heretofore been contemplated in the art. As appreciated by those skilled in the art, active ingredients or medicaments may take the physical form of a solid (e.g. pill, tablet, capsule (both hard and soft elastic), powder, granulation, flakes, troches (lozenges and pastilles), suppositories and semi-solid ointments, pastes, emulsions and creams), a liquid (e.g., solution, spirits, elixir, syrups, sprays and fluid extracts), a gas or a dispersion. A dispersion is a system in which a dispersed phase is distributed through a continuous phase (e.g., aerosols (liquid or solid in gas), suspensions (solid in liquid), emulsion (liquid in liquid), foam (gas in liquid), solid foam (solid in gas) or gel (liquid or solid in solid)). Dispersions can be classified as molecular, colloidal and coarse, depending on size. In many circumstances the different physical forms or phases of more than one active ingredient or medicament may not, however, be suitably combined or mixed together without altering their individual desirable properties, shelf-life, consistency, potency and the like. Providing active ingredients or medicaments in separate capsules may also be undesirable, since it increases the number of capsules a patient or consumer would need to handle and take. Thus, it would be desirable, to provide multi-compartment capsular delivery apparatus and methods that provide active ingredients or medicaments having diverse physical properties (e.g., solid, liquid, gas or dispersion), which may or may not be properly combined or stored together, into a unitary structure (i.e., a multi-compartment capsule) for usage in a single dosage form. Such novel apparatus and methods are disclosed and claimed herein.

### BRIEF SUMMARY AND OBJECTS OF THE INVENTION

In view of the foregoing, it is a primary object of the present invention to provide novel integrated capsule delivery apparatus and methods for delivering diverse physical states (e.g., solid, liquid, gas or dispersion) of a single active ingredient or medicament, or a plurality of active ingredients or medicaments, in a single dosage form, wherein at least two of the active ingredients or medicaments have physical states that differ.

It is also an object of the present invention to provide novel integrated capsule delivery apparatus and methods which facilitate various desirable properties including, for example, controlling time-release of key active ingredients or medicaments, prolonging shelf-life of the active ingredients or medicaments, improving palatability, reducing overall production costs and, accordingly, reducing the number of capsules consumed by a patient or consumer as nutritional or therapeutic agents.

Further, it is an object of the present invention to provide novel integrated capsule delivery apparatus and methods for delivering one or more active ingredients or medicaments (e.g., pharmaceutical, biotechnical, nutraceutical, vitamin, dietary supplement, mineral or combination thereof) in the form of a single dosage, multi-compartment capsule having one or more active ingredients in a primary capsule, and one or more active ingredients introduced into a secondary smaller capsule having a size sufficient for being selectively positionable within the primary capsule, wherein the active ingredient(s) within the primary capsule comprises a physical state (e.g., solid, liquid, gas or dispersion) that is different from the physical state of the active ingredient(s) in the secondary capsule.

It is an additional object of the present invention to provide novel integrated capsule delivery apparatus and methods for delivering one or more active ingredients or medicaments (e.g., pharmaceutical, biotechnical, nutraceutical, vitamin, dietary supplement, mineral or combination thereof) in the form of a single dosage, multi-compartment capsule having one or more active ingredients in a primary capsule and the same active ingredient(s) introduced into a smaller secondary capsule having a size sufficient for being positionable within the primary capsule, wherein the active ingredient(s) in the primary capsule comprises a physical state (e.g., solid, liquid, gas or dispersion) different from the active ingredient(s) in the secondary capsule.

It is a further object of the present invention to provide novel integrated capsule delivery apparatus and methods for delivering one or more active ingredients or medicaments (e.g., pharmaceutical, biotechnical, nutraceutical, vitamin, dietary supplement, mineral or combination thereof) in the form of a single dosage, multi-compartment capsule wherein at least one of the primary and secondary capsules include a time-release coating for controlling the release of the active ingredient(s) contained therein.

It is also another object of the present invention to provide novel integrated capsule delivery apparatus and methods for delivering one or more active ingredients or medicaments (e.g., pharmaceutical, biotechnical, nutraceutical, vitamin, dietary supplement, mineral or combination thereof) in the form of a single dosage, multi-compartment capsule having one or more active ingredients in the capsular body, wherein the capsule includes a longitudinally extending body and at least one dividing wall formed along a length of the extending body to form a first chamber and an opposing second chamber within the capsular body and introducing at least one active ingredient or medicament having a first physical state into the first chamber and at least one active ingredient or medicament having a second physical state into a second chamber, whereas the physical state (e.g., solid, liquid, gas or dispersion) of the ingredient(s) in the first chamber is different from the physical state of the ingredient(s) in the second chamber.

It is an additional object of the present invention to provide novel integrated capsule delivery apparatus and methods for delivering one or more active ingredients or medicaments (e.g., pharmaceutical, biotechnical, nutraceutical, vitamin, dietary supplement, mineral or combination thereof) in the form of a single dosage, multi-compartment capsule having a longitudinally extending body and one or more dividing walls disposed along the length of the longitudinally extending body of the capsule, wherein the capsule and one or more of the dividing walls contained therein may include time-release coatings for controlling the release of the active ingredients or medicaments contained therein, respectively.

It is a further object of the present invention to provide novel integrated capsule delivery apparatus and methods for delivering one or more active ingredients or medicaments (e.g., pharmaceutical, biotechnical, nutraceutical, vitamin, dietary supplement, mineral or combination thereof) in the form of a single dosage, multi-compartment capsule having a plurality of active ingredients or medicaments having the physical form of a solid (e.g., pill, tablet, capsule (both hard and soft elastic), powder, granulation, flakes, troches (lozenges and pastilles), suppositories and semi-solid ointments, pastes, emulsions and creams), a liquid (e.g., solution, spirits, elixir and fluid extracts), a gas or a dispersion (e.g., aerosols (liquid or solid in gas), suspensions (solid in liquid), emulsion (liquid in liquid), foam (gas in liquid), solid foam (solid in gas) or gel (liquid or solid in solid), wherein the physical form of the active ingredients differ between a primary and secondary capsule, and between one or more dividing walls disposed in spaced-apart relationship along the length of a longitudinally extending capsular body.

It is a still further object of the present invention to provide novel integrated capsule delivery apparatus and methods for delivering one or more active ingredients or medicaments (e.g., pharmaceutical, biotechnical, nutraceutical, vitamin, dietary supplement, mineral or combination thereof) in the form of a single dosage, multi-compartment capsule, wherein an encapsulation process comprises the steps of: (1) providing a capsule comprising a first end, a second end, a longitudinally extending body having a length disposed between the first and second ends, and a plurality of dividing walls spaced apart along the length of the extending body, wherein the dividing walls form a plurality of receiving chambers; (2) introducing at least one active ingredient having a first physical state into a first receiving chamber; (3) introducing at least one active ingredient having a second physical state into a second receiving chamber; (4) introducing at least one active ingredient having a third physical state into a third receiving chamber, wherein the physical states of at least two of the active ingredients introduced into the first, second or third receiving chambers differ; and (5) sealing the first and second ends of said capsule.

Additionally, it is an object of the present invention to provide novel integrated capsule delivery apparatus and methods for delivering a single dosage, multi-compartment capsule comprising a capsular base and cap configuration, wherein the size and shape of the cap, relative to its sealing relationship with the base, generally eliminates or substantially reduces any potential dead space volume within the internal periphery of the capsule, thereby functionally negating the opportunity for reaction between an air bubble and one or more active ingredients introduced into the capsule and, accordingly, improving stability of the capsular ingredient(s).

Consistent with the foregoing objects, and in accordance with the invention as embodied and broadly described herein, one presently preferred embodiment of the novel integrated capsule delivery apparatus and methods of the present invention comprises a multi-compartment capsule including a primary capsule and a secondary capsule selectively positionable within an internal periphery of the primary capsule. The secondary capsule may include a base, a corresponding cap and one or more receiving chambers. Each of the receiving chambers of the secondary capsule may be formed having an internal periphery sufficient for receiving at least one active ingredient or medicament (e.g., pharmaceutical, biotechnical, nutraceutical, vitamin, dietary supplement, mineral or combination thereof) therein. Similarly, the primary capsule may be formed having a base, a corresponding cap and one or more receiving chambers. The receiving chambers of the primary capsule may be formed having an internal periphery sufficient for receiving the secondary capsule and one or more active ingredients or medicaments (e.g., pharmaceutical, biotechnical, nutraceutical, vitamin, dietary supplement, mineral or combination thereof) having a physical state (i.e., solid, liquid, gas or dispersion) different from the physical state of the active ingredient(s) housed within the receiving chamber of the secondary capsule.

As further contemplated herein, a multi-compartment capsule is provided comprising a base, a corresponding cap and one or more dividing walls positionable between the base and the cap. Structurally, the size, shape and positioning of the dividing walls relative to the base and corresponding cap facilitates the formation of at least two, independent and separate receiving chambers. Each of the receiving chambers having an internal periphery sufficient for receiving one or more active ingredients or medicaments (e.g., pharmaceutical, biotechnical, nutraceutical, vitamin, dietary supplement, mineral or combination thereof) therein. In preferred design, the physical state (e.g., solid, liquid, gas or dispersion) of the active ingredient(s) in the first receiving chamber is different from the physical state of the active ingredient(s) in the second receiving chamber. After introducing one or more active ingredients or medicaments into each receiving chamber, the cap may be selectively positioned in sealing relationship with the base to form one presently preferred embodiment of the single, dosage multi-compartment capsule.

One presently preferred embodiment of an encapsulation process for forming a multi-compartment capsule may comprise the steps of: (1) providing a primary capsule having a base, a corresponding cap and a receiving chamber; (2) providing a secondary capsule having a base, a corresponding cap and a receiving chamber; (3) introducing at least one ingredient or medicament (e.g., pharmaceutical, biotechnical, nutraceutical, vitamin, dietary supplement, mineral or combination thereof) having a first physical state (e.g., solid, liquid, gas or dispersion) into at least a portion of the receiving chamber of the secondary capsule and selectively positioning the cap in sealing relationship with the base; (4) introducing at least one ingredient or medicament (e.g., pharmaceutical, biotechnical, nutraceutical, vitamin, dietary supplement, mineral or combination thereof) having a second physical state (e.g., solid, liquid, gas or dispersion) into at least a portion of the receiving chamber of the primary capsule, wherein the first physical state of the ingredient(s) in the secondary capsule is different from the second physical state of the ingredient(s) in the primary capsule; and (5) introducing the secondary capsule into at least a portion of the receiving chamber of the primary capsule and selectively positioning the cap in sealing relationship with the base to form a single dosage multi-compartment capsule.

In alternate presently preferred embodiments of the present invention, a tertiary capsule comprising a base, a corresponding cap and a receiving chamber having an internal periphery sufficient for receiving one or more active ingredients or medicaments (e.g., pharmaceutical, biotechnical, nutraceutical, vitamin, dietary supplement, mineral or combination thereof) may be selectively introduced within an internal periphery of at least one receiving chamber of the secondary capsule. After the introduction of at least one active ingredient into one or more receiving chambers of a tertiary capsule pursuant to an encapsulation process of the present invention, the cap of the tertiary capsule may be selectively positioned in sealing relationship with the base and then introduced into at least a portion of the internal periphery of the secondary capsule, together with one or more active ingredients therein. It is contemplated herein that at least two of the active ingredients introduced within the receiving chambers of the primary, secondary and tertiary capsules, respectively, comprise at least two different physical states (e.g., solid, liquid, gas or dispersion).

In preferred structural design, the primary capsule may comprise a cap having a generally U-shaped configuration adapted to provide a sealing relationship when engaging the corresponding base, thereby reducing dead space volume in the internal periphery of the cap and receiving chamber of the base. A cap having a configuration adapted to generally eliminate or substantially reduce potential dead space volume of the cap and receiving chamber of the base may, accordingly, function to negate the potential for a reaction between an air bubble and one or more active ingredient(s) introduced into the base of the primary capsule.

Alternatively, a multi-compartment capsule of the present invention may include the introduction of a filling material into the cap of the primary capsule, the cap having a general cylindrical configuration adapted to provide a sealing relationship when engaging the corresponding base. An amount of filling material may be introduced into at least a portion of the internal periphery of the cap to fill, either partially or completely, the inner volume of the cap, thereby reducing the dead space volume in the cap and the internal periphery of the receiving chamber of the base. In this regard, the introduction of a filling material relative to the internal periphery of the cap may generally eliminate or substantially reduce the potential dead space volume, thus functionally negating the potential for a reaction between an air bubble and one or more active ingredient(s) introduced into the base of the primary capsule.

The primary, secondary or tertiary capsules, in accordance with the present invention, may be formed having the same or different colors. Moreover, the base and corresponding cap of a single capsule may be formed having different colors in an effort to enhance the aesthetics of the capsule to the consumer. In one presently preferred embodiment of a multi-compartment capsule of the present invention, the dosage may be banded, sealed or easily dividable in a contact area of the primary and secondary capsules or the sealing band may be color-coded to assist in branding, if desired.

It is further contemplated herein that a multi-compartment capsule of the present invention may comprise component parts of the capsule having various time-release coatings to facilitate the release and ultimately the absorption of those active ingredients introduced into the different receiving chambers of the multi-compartment capsule to release at different release rates. In particular, a primary capsule may be formed having a conventional time-release coating that dissolves and releases the active ingredient(s) contained therein before the timed-release of the active ingredient(s) contained within a secondary capsule. Likewise, the dividing walls disposed within the internal periphery of the base of a capsule may be formed having conventional time-release coatings that dissolve and release the active ingredients within each receiving chamber defined by the dividing walls at different rates, thereby delivering the active ingredients or medicaments contained within a multi-compartment capsule at different rates. Certain active ingredients or medicaments may, therefore, be delivered at a selected interval, while other ingredients may be released at a later interval. In this way, the novel design of the multi-compartment capsules of the present invention may facilitate precision delivery of active ingredients to targeted areas of the consumer.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a flow diagram illustrating one presently preferred embodiment of a process of the present invention comprising the steps of introducing at least one active ingredient or medicament having a solid physical state into a secondary capsule and introducing the secondary capsule into a primary capsule further including at least one active ingredient or medicament having a liquid physical state;

FIG. 2 is a cross-sectional view illustrating another presently preferred embodiment of a multi-compartment capsule of the present invention wherein a primary capsule houses a secondary capsule and a secondary capsule houses a tertiary capsule, wherein each of the capsules include one or more active ingredients or medicaments and the active ingredient(s) introduced into at least two of the capsules comprise different physical states;

FIG. 3 is a perspective view illustrating yet another presently preferred embodiment of a multi-compartment capsule comprising a base, a cap and a dividing wall positioned between the base and the cap, wherein the dividing wall facilitates the formation of at least two, independent receiving chambers for receiving one or more active ingredients or medicaments having different physical states;

FIG. 4 is a cross-sectional view of the multi-compartment capsule shown in FIG. 3 wherein the base, the dividing wall defining the two receiving chambers and the cap are assembled to form a capsule of the present invention and wherein one or more active ingredients or medicaments having different physical states are introduced into the receiving chambers;

FIG. 5 is a perspective view illustrating an alternate presently preferred embodiment of a multi-compartment capsule of the present invention having a primary capsule comprising a capsular base configured with a longitudinally extending body, a corresponding cap and a series of dividing walls disposed in spaced apart relationship along the length of the longitudinally extending body of the base, wherein the dividing walls define a plurality of independent receiving chambers having an internal periphery sufficient for introducing one or more active ingredients or medicaments having different physical states therein and for introducing a secondary capsule, having one or more active ingredients contained therein, within at least one of said receiving chambers;

FIG. 6 is a cross-sectional view of the multi-compartment capsule shown in FIG. 5 wherein the base and the cap are assembled to form a single dosage capsule having a series of dividing walls that define a plurality of chambers for receiving one or more active ingredients or medicaments, wherein the active ingredient(s) in at least two of the receiving chambers comprise different physical states;

FIG. 7 is a perspective view illustrating yet another presently preferred embodiment of a multi-compartment capsule of the present invention having a primary capsule comprising a capsular base configured with a longitudinally extending body, a corresponding cap and a series of dividing walls disposed in spaced apart relationship, both vertically and horizontally, along the length of the longitudinally extending body of the base, wherein the dividing walls define a plurality of independent receiving chambers having an internal periphery sufficient for introducing one or more active ingredients or medicaments having different physical states therein;

FIG. 8 is a perspective view illustrating an alternate preferred embodiment of the multi-compartment capsule shown in FIG. 7, wherein the multi-compartment capsule includes a primary capsule comprising a capsular base configured with a longitudinally extending body, a corresponding cap and a series of dividing walls disposed in spaced apart relationship, both vertically and horizontally, along the length of the longitudinally extending body of the base, wherein the dividing walls define a plurality of independent receiving chambers having an internal periphery sufficient for introducing one or more active ingredients or medicaments having different physical states therein and for introducing a secondary capsule, having one or more active ingredients contained therein, within at least one of said receiving chambers;

FIG. 9 is a perspective view illustrating yet another presently preferred embodiment of a multi-compartment capsule of the present invention wherein the multi-compartment capsule shown in FIG. 7 is introduced within the internal periphery of a receiving chamber of a primary capsule having one or more active ingredients also contained therein;

FIG. 10 is a cross-sectional view illustrating a presently preferred embodiment of a multi-compartment capsule of the present invention including a secondary capsule having one or more active ingredients or medicaments selectively introduced into the internal periphery of a primary capsule having one or more active ingredients or medicaments, wherein the active ingredient(s) introduced into the primary capsule comprises a physical state (e.g., solid, liquid, gas or dispersion) which differs from the physical state of the active ingredient(s) introduced into the internal periphery of the secondary capsule, the primary capsule further comprising a cap having a generally U-shaped configuration adapted to provide a sealing relationship when engaging the corresponding base, thereby reducing dead space volume in the internal periphery of the receiving chamber of the base;

FIG. 11 is a perspective view illustrating yet another presently preferred embodiment of a multi-compartment capsule of the present invention including a secondary capsule having one or more active ingredients or medicaments and having a size and shape sufficient for being selectively introduced into the internal periphery of a primary capsule having one or more active ingredients or medicaments, wherein the active ingredient(s) introduced into the primary capsule comprises a physical state (e.g., solid, liquid, gas or dispersion) which differs from the physical state of the active ingredient(s) introduced into the internal periphery of the secondary capsule, the primary capsule further comprising a filling material introduced into the internal periphery of the cap having a general conical configuration and adapted to provide a sealing relationship when engaging the corresponding base, thereby reducing dead space volume in the internal periphery of the receiving chamber of the base;

FIG. 12 is a cross-sectional view of the multi-compartment capsule shown in FIG. 11 wherein a sufficient amount of filling material is introduced into the internal periphery of the cap, thereby functioning to eliminate or significantly reduce the dead space volume in the receiving chamber of the primary capsule; and

FIG. 13 is a cross-sectional view illustrating an alternate presently preferred embodiment of a multi-compartment capsule of the present invention comprising a tertiary capsule having one or more active ingredients or medicaments and having a size a shape sufficient for being introduced into at least a portion of the internal periphery of the receiving chamber of a secondary capsule having one or more active ingredients or medicaments also introduced therein, the size and shape of the secondary capsule sufficient for being selectively introduced into the internal periphery of a primary capsule having one or more active ingredients or medicaments, wherein the active ingredient(s) introduced into the primary capsule comprises a physical state (e.g., solid, liquid, gas or dispersion) which differs from the physical state of the active ingredient(s) introduced into the receiving chambers of the secondary and tertiary capsules, the primary capsule further comprising a filling material introduced into the internal periphery of the cap having a general conical configuration and adapted to provide a sealing relationship when engaging the corresponding base, thereby reducing dead space volume in the internal periphery of the receiving chamber of the base of the primary capsule.

### DETAILED DESCRIPTION OF THE PREFERRED EMBODIMENTS

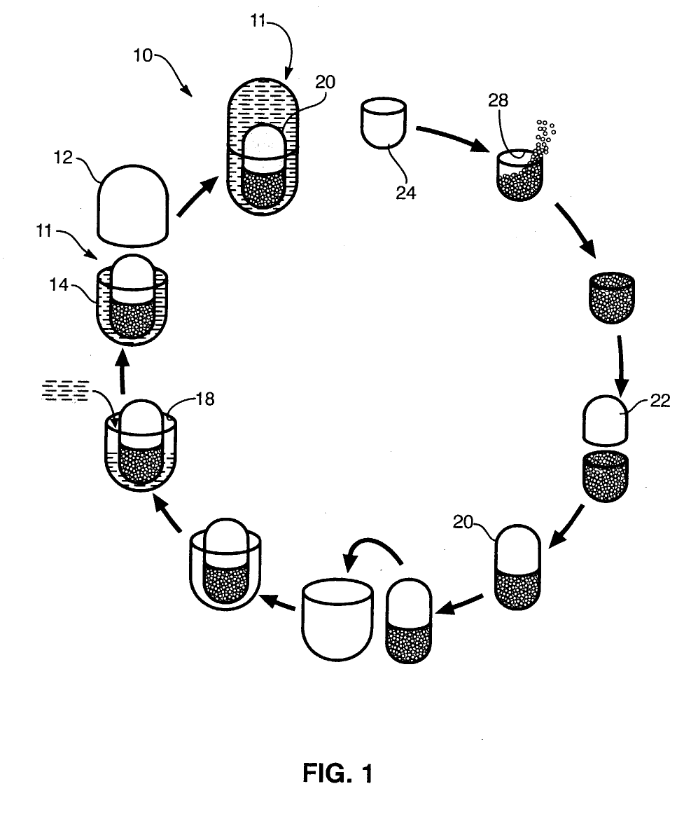

It will be readily understood that the components of the present invention, as generally described and illustrated in the Figures herein, could be arranged and designed in a wide variety of different configurations and process steps. Those of ordinary skill in the art will, of course, appreciate that various modifications to the details herein may easily be made without departing from the essential characteristics of the invention, as described. Thus, the following more detailed description of the embodiments of apparatus and methods of the present invention, as represented in FIGS. 1 through 13, is not intended to limit the scope of the invention, as claimed, but it is merely representative of the presently preferred embodiments of the invention.

The presently preferred embodiments of the invention will be best understood by reference to the drawings, wherein like parts are designated by like numerals throughout.

One presently preferred embodiment of the present invention, designated generally at 10, is best illustrated in FIG. 1. As shown, a multi-compartment capsule 10 is illustrated comprising a primary capsule 11 and a secondary capsule 20 selectively introduced within at least a portion of an internal periphery of the primary capsule. The secondary capsule 20 includes a base 24, a corresponding cap 22 and a receiving chamber 28 formed between the base and cap. The receiving chamber 28 is configured having an internal periphery sufficient for receiving at least one active ingredient or medicament (e.g., pharmaceutical, biotechnical, nutraceutical, vitamin, dietary supplement, mineral or combination thereof) therein. In similar structural design, the primary capsule 11 may be formed having a base 14, a corresponding cap 12 and a receiving chamber 18 formed between the base and cap. The receiving chamber 18 of the primary capsule 11 is preferably formed having an internal periphery sufficient for receiving the secondary capsule 20, together with at least one active ingredient or medicament (e.g., pharmaceutical, biotechnical, nutraceutical, vitamin, dietary supplement, mineral or combination thereof) therein.

Still referring to FIG. 1, one presently preferred embodiment of an encapsulation process for forming a multi-compartment capsule 10 is comprising the steps of: (1) providing a primary capsule 11 having a base 14, a corresponding cap 12 and a receiving chamber 18; (2) providing a secondary capsule 20 having a base 24, a corresponding cap 22 and a receiving chamber 28; (3) introducing at least one ingredient or medicament (e.g., pharmaceutical, biotechnical, nutraceutical, vitamin, dietary supplement, mineral or combination thereof) having a first physical state (e.g., solid, liquid, gas or dispersion) into at least a portion of the receiving chamber 28 of the secondary capsule 20 and selectively positioning the cap 22 in sealing relationship with the base 24; (4) introducing at least one ingredient or medicament (e.g., pharmaceutical, biotechnical, nutraceutical, vitamin, dietary supplement, mineral or combination thereof) having a second physical state (e.g., solid, liquid, gas or dispersion) into at least a portion of the receiving chamber 18 of the primary capsule 11, wherein the first physical state of the ingredient(s) in the secondary capsule is different from the second physical state of the ingredient(s) in the primary capsule; and (5) introducing the secondary capsule 20 into at least a portion of the receiving chamber 18 of the primary capsule 11 and selectively positioning the cap 12 in sealing relationship with the base 14 to form a single dosage multi-compartment capsule.

As shown, a solid is selectively introduced within at least a portion of the internal periphery of the receiving chamber 28 of the secondary capsule 20 and a liquid is selectively introduced within at least a portion of the internal periphery of the receiving chamber 18 of the primary capsule 11. Although the ingredient(s) introduced into the receiving chamber 18 of the primary capsule 11 may be the same or different from the ingredient(s) introduced into the receiving chamber 28 of the secondary capsule, the active ingredient(s) in the primary capsule 11 have a physical state (i.e., solid, liquid, gas or dispersion) that varies from the physical state of the active ingredient(s) in the secondary capsule 20. Accordingly, those skilled in the art will readily recognize other possible modifications and adaptations relative to the contemplated variations in physical states of the active ingredient(s) selectively positionable within the receiving chambers 18, 28 of the primary and secondary capsules, respectively, which are consistent with the spirit and scope of the present invention. It is intended, therefore, that the figures and examples provided herein be viewed as exemplary of the principles of the present invention, and not as restrictive to a particular structure or method for implementing those principles.

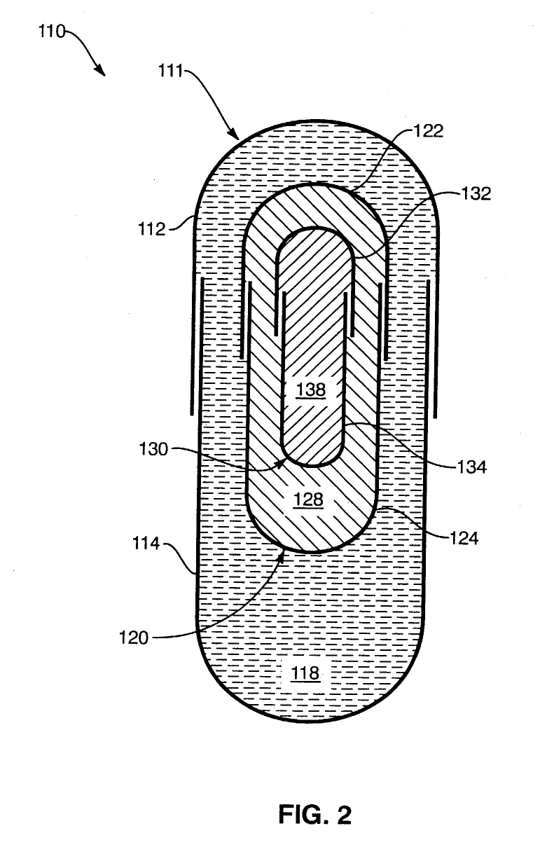

Referring now to FIG. 2, an alternate presently preferred embodiment of a multi-compartment capsule 110 is shown comprising a primary capsule 111, a secondary capsule 120 and a tertiary capsule 130. The tertiary capsule 130 includes a base 134, a corresponding cap 132 and a receiving chamber 138 formed between the base and cap. The receiving chamber 138 is preferably formed having an internal periphery sufficient for receiving at least one active ingredient or medicament (e.g., pharmaceutical, biotechnical, nutraceutical, vitamin, dietary supplement, mineral or combination thereof). Structurally, the tertiary capsule 130 is configured having a size sufficient for being selectively introduced within at least a portion of an internal periphery of a receiving chamber 128 defined between a base 124 and a corresponding cap 122 of the secondary capsule 120. One or more active ingredients or medicaments (e.g., pharmaceutical, biotechnical, nutraceutical, vitamin, dietary supplement, mineral or combination thereof) may be introduced into at least a portion of the receiving chamber 128 of the secondary capsule 120, together with the introduction of the tertiary capsule 130 comprising its active ingredient(s). The secondary capsule 120 having its active ingredient(s) and housing the tertiary capsule 130 with its active ingredient(s) may then be selectively introduced within at least a portion of an internal periphery of a receiving chamber 118 of the primary capsule 111 defined between a base 124 and a corresponding cap 122. Preferably, the receiving chamber 118 of the primary capsule 111 may also include one or more active ingredients or medicaments (e.g., pharmaceutical, biotechnical, nutraceutical, vitamin, dietary supplement, mineral or combination thereof) introduced therein.

Still referring to FIG. 2, another presently preferred embodiment of an encapsulation process for forming a multi-compartment capsule 110 may comprise the steps of: (1) providing a primary capsule 111 having a base 114, a corresponding cap 112 and a receiving chamber 118 defined between the base and cap; (2) providing a secondary capsule 120 having a base 124, a corresponding cap 122 and a receiving chamber 128 defined between the base and cap; (3) providing a tertiary capsule 130 having a base 134, a corresponding cap 132 and a receiving chamber 138 defined between the base and cap; (4) introducing at least one ingredient (e.g., pharmaceutical, biotechnical, nutraceutical, vitamin, dietary supplement, mineral or combination thereof) having a first physical state (e.g., solid, liquid, gas or dispersion) into at least a portion of the receiving chamber 138 of the tertiary capsule 130 and selectively positioning the cap 132 in sealing relationship with the base 134; (5) introducing at least one ingredient (e.g., pharmaceutical, biotechnical, nutraceutical, vitamin, dietary supplement, mineral or combination thereof) having a second physical state (e.g., solid, liquid, gas or dispersion) into at least a portion of the receiving chamber 128 of the secondary capsule 120, wherein the first physical state of the ingredient(s) in the tertiary capsule 130 are the same as the second physical state of the ingredient(s) in the secondary capsule 120; (6) introducing the tertiary capsule 130 into at least a portion of the receiving chamber 218 of the secondary capsule 120 and selectively positioning the cap 122 in sealing relationship with the base 124; (7) introducing at least one ingredient (e.g., pharmaceutical, biotechnical, nutraceutical, vitamin, dietary supplement, mineral or combination thereof) having a third physical state (e.g., solid, liquid, gas or dispersion) into at least a portion of the receiving chamber 118 of the primary capsule 111, wherein the third physical state of the ingredient(s) in the primary capsule are different from the first and second physical states of the ingredient(s) in the tertiary capsule 130 and the secondary capsule 120, respectively; and (8) introducing the secondary capsule 120 into at least a portion of the receiving chamber 118 of the primary capsule 111 and selectively positioning the cap 112 in sealing relationship with the base 114 to form a single dosage multi-compartment capsule.

In the presently preferred embodiment illustrated in FIG. 2, a liquid may be selectively introduced into at least a portion of the internal periphery of the receiving chamber 118 of the primary capsule 111, a solid may be selectively introduced into at least a portion of the internal periphery of the receiving chamber 128 of the secondary capsule 120 and a solid may be selectively introduced into at least a portion of the receiving chamber 138 of the tertiary capsule 130. Although the ingredient(s) selectively introduced into the receiving chambers 118, 128, 138 of the primary, secondary and tertiary capsules 111, 120, 130, respectively, may be the same or different, the active ingredient(s) in at least two of the receiving chambers comprise at least two different physical states (e.g., solid, liquid, gas or dispersion). It is further contemplated herein as an alternate embodiment that the active ingredient(s) introduced in the receiving chamber 118 of the primary capsule 111 comprises a physical state (e.g., solid, liquid, gas or dispersion) different from the physical state of the active ingredient(s) contained within the receiving chamber 128 of the secondary capsule 120 which is different from the physical state of the active ingredient(s) contained within the receiving chamber 138 of the tertiary capsule 130. Those skilled in the art will readily recognize other possible modifications and adaptations relative to contemplated variations in physical states of the active ingredient(s) selectively introduced within the receiving chambers 118, 128, 138 of the primary, secondary and tertiary capsules, respectively, which are consistent with the spirit and scope of the present invention. It is intended, therefore, that the figures and examples provided herein be viewed as exemplary of the principles of the present invention, and not as restrictive to a particular structure or method for implementing those principles.

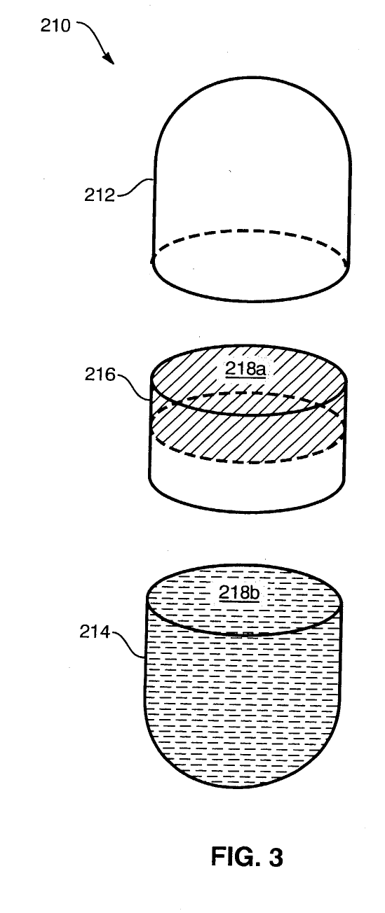

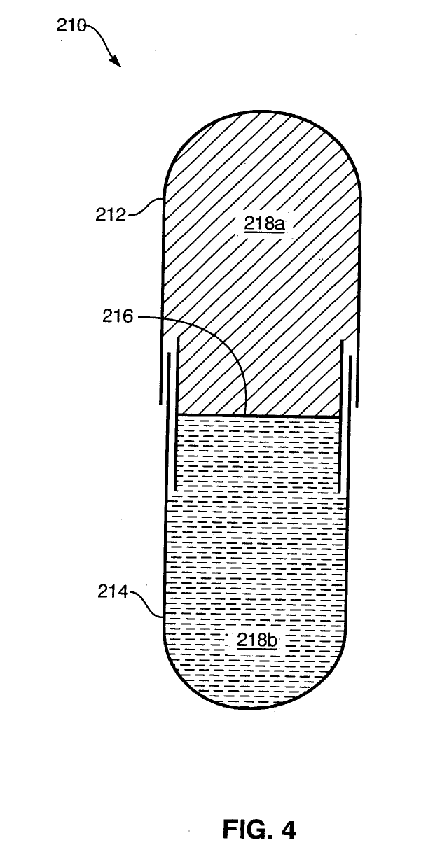

Referring now to FIGS. 3 and 4, another presently preferred embodiment of a multi-compartment capsule 210 is shown comprising a base 214, a corresponding cap 212 and a dividing wall 216 positionable between the base and the cap. Structurally, the size, shape and positioning of the dividing wall 216 relative to the base 214 and corresponding cap 212 facilitates the formation of at least two, independent and separate receiving chambers 218a , 218b, each having an internal periphery sufficient for receiving one or more active ingredients or medicaments (e.g., pharmaceutical, biotechnical, nutraceutical, vitamin, dietary supplement, mineral or combination thereof) therein. As best shown in FIG. 4, the dividing wall 216 seats within the internal periphery of both the base 214 and the corresponding cap 212. After introducing one or more active ingredients or medicaments into receiving chamber 218b and disposing the dividing wall 216 relative thereto, one or more active ingredients or medicaments (e.g., pharmaceutical, biotechnical, nutraceutical, vitamin, dietary supplement, mineral or combination thereof) may be introduced into receiving chamber 218a and the cap may be selectively positioned in sealing relationship with the base 214 to form one presently preferred embodiment of the single, dosage multi-compartment capsule 210. Moreover, the dividing wall 216 may functionally assist in forming a sealing relationship between the base 214 and corresponding cap 212 of the multi-compartment capsule 210, if desired.

In one presently preferred embodiment of the multi-compartment capsule 211 of the present invention, a solid may be selectively introduced into at least a portion of the internal periphery of the receiving chamber 218a and a liquid may be selectively introduced into at least a portion of the internal periphery of the receiving chamber 218b. Although the ingredient(s) introduced into the receiving chamber 218a may be the same or different from the ingredient(s) introduced into the receiving chamber 218, the active ingredient(s) in the first receiving chamber 218a preferably comprise a physical state (e.g., solid, liquid, gas or dispersion) that is different from the physical state of the active ingredient(s) in the second receiving chamber 218b. Those skilled in the art will readily recognize other possible modifications and adaptations relative to the contemplated variations in physical states (e.g., solid, liquid, gas and dispersion) of the active ingredient(s) selectively positionable within the receiving chambers 218a, 218b which are consistent with the spirit and scope of the present invention. It is intended, therefore, that the figures and examples provided herein be viewed as exemplary of the principles of the present invention, and not as restrictive to a particular structure or method for implementing those principles.

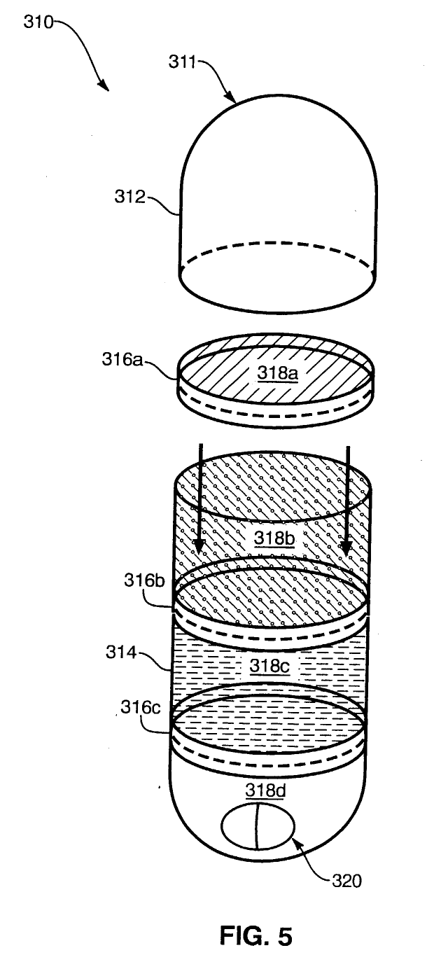

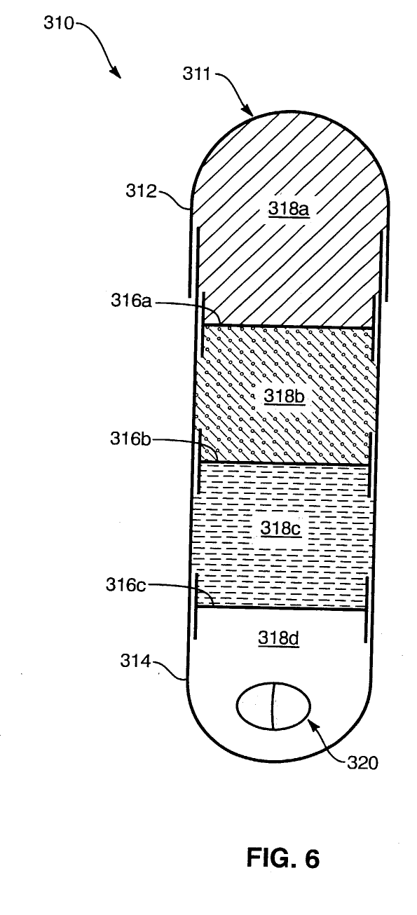

Referring now to FIGS. 5 and 6, another presently preferred embodiment of a multi-compartment capsule, designated as 310, is shown including a primary capsule 311 comprising a capsular base 314 configured having an elongated or longitudinally extending body, a corresponding cap 312 and a plurality of dividing walls 316 selectively disposed along the length of the longitudinally extending body of the base. Preferably, the structural size, shape and positioning of the dividing walls 316a, 316b, 316c along the length of the elongated body of the base 314 facilitate the formation of a plurality of independent receiving chambers 318a, 318b, 318c, 318d. Each receiving chamber 318a, 318b, 318c, 318d of the primary capsule 311 having an internal periphery sufficient for receiving one or more active ingredients or medicaments (e.g., pharmaceutical, biotechnical, nutraceutical, vitamin, dietary supplement, mineral or combination thereof) therein.

As best shown in FIG. 6, the dividing walls 316a, 316b, 316c are preferably seated within the internal periphery of the base 314 of the primary capsule 311 and in a spaced apart relationship along the length of the longitudinally extending body and form four independent receiving chambers 318a, 318b, 318c, 318d. In one presently preferred embodiment of the multi-compartment capsule 310 of the present invention, each of the receiving chambers 318a, 318b, 316c comprises at least one active ingredient or medicament having a physical state (e.g., solid, liquid, gas or dispersion) different from the physical state of the ingredient(s) in the other receiving chambers.

As illustrated by way of example, and not by way of restriction, a solid may be selectively introduced into at least a portion of the internal periphery of the receiving chamber 318a, a dispersion may be selectively introduced into at least a portion of the internal periphery of the receiving chamber 318b, a liquid may be selectively introduced into at least a portion of the internal periphery of the receiving chamber 316c and a secondary capsule 320 may be selectively introduced into at least a portion of the internal periphery of the receiving chamber 318d. As contemplated herein, receiving chamber 318d may be further configured having an internal periphery sufficient for receiving a secondary capsule 320, together with at least one active ingredient or medicament therein.

One presently preferred embodiment of an encapsulation process, as defined by the structural configuration of the multi-compartment capsule 310 illustrated in FIGS. 5 and 6, may comprise the steps of: (1) introducing a secondary capsule 320 (e.g., tablet) and one or more active ingredients or medicaments into receiving chamber 318d; (2) selectively positioning dividing wall 316c along the length of the elongated body of the base 314; (3) introducing one or more active ingredients or medicaments (e.g., pharmaceutical, biotechnical, nutraceutical, vitamin, dietary supplement, mineral or combination thereof) into receiving chamber 318c; (4) selectively positioning dividing wall 316b along the length of the elongated body of the base 314 in a spaced apart relationship to dividing wall 316c; (5) introducing one or more active ingredients or medicaments (e.g., pharmaceutical, biotechnical, nutraceutical, vitamin, dietary supplement, mineral or combination thereof) into receiving chamber 318b; (6) selectively positioning dividing wall 316a along the length of the elongated body of the base 314 in a spaced apart relationship to dividing wall 316b;and (7) selectively positioning the cap 312 in sealing relationship with the base 314 to form a presently preferred embodiment of a single, dosage multi-compartment capsule 310. The dividing wall 316a may also function in the formation of the sealing relationship between the base 314 and the corresponding cap 312, if desired.

Although the ingredient(s) introduced into one of the receiving chambers 318 may be the same ingredient or may be different from the ingredient(s) introduced into the other receiving chambers, the active ingredient(s) in at least two of the receiving chambers 318 preferably comprise a physical state (e.g., solid, liquid, gas or dispersion) that is different from the physical state of the active ingredient(s) in one or more of the remaining receiving chambers. Those skilled in the art will readily recognize other possible modifications and adaptations relative to the contemplated variations in physical states (e.g., solid, liquid, gas and dispersion) of the active ingredient(s) selectively introduced within the receiving chambers 318 which are consistent with the spirit and scope of the present invention. It is intended, therefore, that the figures and examples provided herein be viewed as exemplary of the principles of the present invention, and not as restrictive to a particular structure or method for implementing those principles.

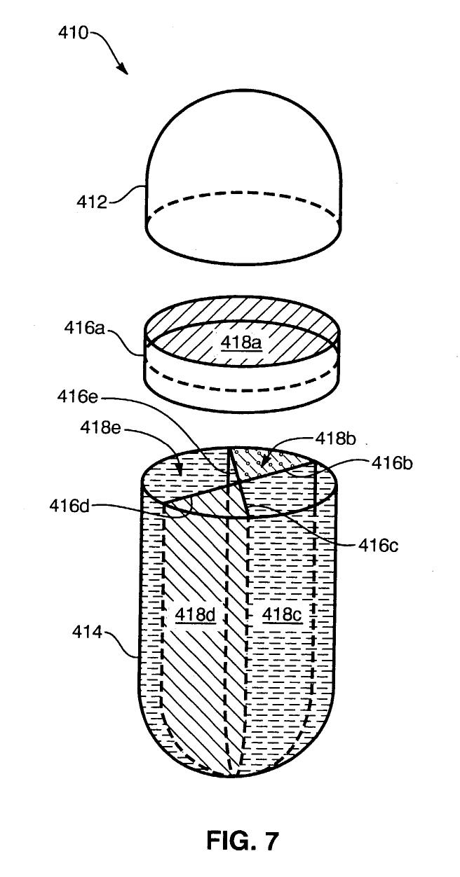

Another presently preferred embodiment of a multi-compartment capsule of the present invention, generally designated as 410 in FIG. 7, is shown comprising a capsular base 414 preferably configured having an elongated or longitudinally extending body, a corresponding cap 412 and a plurality of dividing walls 416 selectively disposed along the length of the longitudinally extending body of the base, both horizontally and vertically. In structural design, the size, shape and positioning of the dividing walls 416a, 416b, 416c, 416d, 416e along the length of the longitudinally extending body of the base 414 facilitate the formation of a plurality of independent receiving chambers 418.

In one presently preferred embodiment, the dividing walls 416a, 416b, 416c, 416d, 416e are preferably disposed or seated in a spaced apart relationship within the internal periphery of the base 414 of the primary capsule 411 along the length of the longitudinally extending body, whereby forming five (5) independent receiving chambers 418a, 418b, 418c, 418d, 418e. Each receiving chamber 418a, 418b, 418c, 418d, 418e of the primary capsule 411 are preferably configured having an internal periphery dimensionally sufficient for receiving one or more active ingredients or medicaments (e.g., pharmaceutical, biotechnical, nutraceutical, vitamin, dietary supplement, mineral or combination thereof) therein.

Still referring to FIG. 7, one presently preferred embodiment of an encapsulation process, as defined by the structural configuration of the multi-compartment capsule 410, may comprise the steps of: (1) introducing one or more active ingredients or medicaments into receiving chamber 418e defined by dividing walls 416d, 416e which are vertically disposed along the length of the elongated body of the base 414; (2) introducing one or more active ingredients or medicaments into receiving chamber 418d defined by dividing walls 416c, 416d which are vertically disposed along the length of the elongated body of the base 414; (3) introducing one or more active ingredients or medicaments into receiving chamber 416c defined by dividing walls 416b, 416c which are vertically disposed along the length of the elongated body of the base 414; (4) introducing one or more active ingredients or medicaments into receiving chamber 418b defined by dividing walls 416b, 416e which are vertically disposed along the length of the elongated body of the base 414; (5) disposing dividing wall 416a along the length of the elongated body of the base 414 perpendicular to the disposition of dividing walls 416b, 416c, 416d, 416e and introducing one or more active ingredients or medicaments into receiving chamber 418a; and (6) selectively positioning the cap 412 in sealing relationship with the base 414 to form one presently preferred embodiment of a single, dosage multi-compartment capsule 410. As appreciated, the dividing wall 416a may also function in the formation of the sealing relationship between the base 414 and the corresponding cap 412, if structurally desired.

As illustrated by way of example, and not by way of restriction, a solid may be selectively introduced into at least a portion of the internal periphery of the receiving chamber 418a, a dispersion may be selectively introduced into at least a portion of the internal periphery of the receiving chamber 418b, a liquid may be selectively introduced into at least a portion of the internal periphery of the receiving chamber 418c, a solid may be selectively introduced into at least a portion of the internal periphery of the receiving chamber 418d and a liquid may be selectively introduced into at least a portion of the internal periphery of the receiving chamber 418e.

Although the ingredient(s) introduced into one of the receiving chambers 418 may be the same ingredient or may be different from the ingredient(s) introduced into the other receiving chambers, the active ingredient(s) in at least two of the receiving chambers 418 preferably comprise a physical state (e.g., solid, liquid, gas or dispersion) that is different from the physical state of the active ingredient(s) in one or more of the remaining receiving chambers. Those skilled in the art will readily recognize other possible modifications and adaptations relative to the contemplated variations in physical states (e.g., solid, liquid, gas and dispersion) of the active ingredient(s) selectively introduced within the receiving chambers 418 which are consistent with the spirit and scope of the present invention. It is intended, therefore, that the figures and examples provided herein be viewed as exemplary of the principles of the present invention, and not as restrictive to a particular structure or method for implementing those principles.

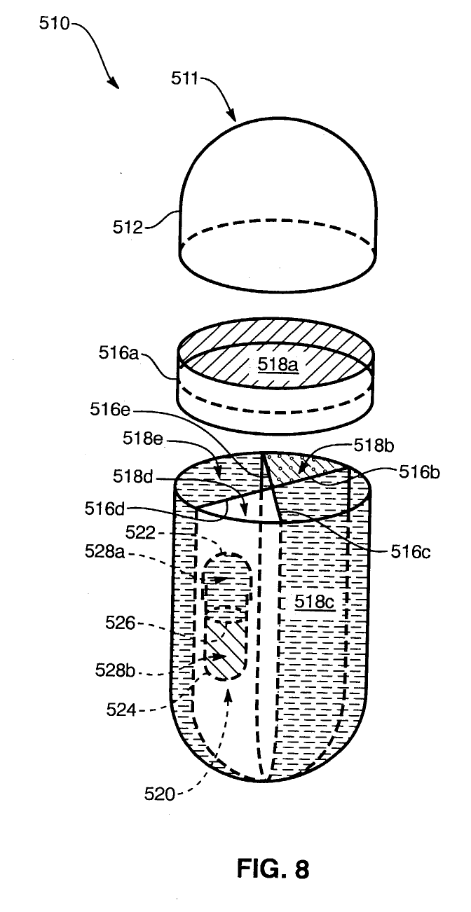

Referring now to FIG. 8, an alternate presently preferred embodiment of a multi-compartment capsule 510 includes a capsular base 514 preferably configured having an elongated or longitudinally extending body, a corresponding cap 512 and a plurality of dividing walls 516 selectively disposed along the length of the longitudinally extending body of the base, both horizontally and vertically. In structural design, the size, shape and positioning of the dividing walls 516a, 516b, 516c, 516d along the length of the longitudinally extending body of the base 514 facilitate the formation of a plurality of independent receiving chambers 518.

In one presently preferred embodiment, the dividing walls 516a, 516b, 516c, 516d, 516e are preferably disposed or seated in a spaced apart relationship within the internal periphery of the base 514 of the primary capsule 511 along the length of the longitudinally extending body, whereby forming five (5) independent receiving chambers 518a, 518b, 518c, 518d, 518e. Each of the receiving chamber 518a, 518b, 518c, 518d, 518e of the primary capsule 411are preferably configured having an internal periphery dimensionally sufficient for receiving one or more active ingredients or medicaments (e.g., pharmaceutical, biotechnical, nutraceutical, vitamin, dietary supplement, mineral or combination thereof) therein. Moreover, receiving chamber 518d is formed having an internal periphery sufficient for receiving a secondary capsule 520. The secondary capsule 520 being configured with a base 524, corresponding cap 522 and a dividing wall 526 defining a first receiving chamber 528a and a second receiving chamber 528b. The first receiving chamber 528a is preferably configured having an internal periphery sufficient for receiving one or more active ingredients or medicaments (e.g., pharmaceutical, biotechnical, nutraceutical, vitamin, dietary supplement, mineral or combination thereof) having a first physical state (e.g., solid, liquid, gas or dispersion) therein. Similarly, the second receiving chamber 528b is configured having an internal periphery sufficient for receiving one or more active ingredients or medicaments (e.g., pharmaceutical, biotechnical, nutraceutical, vitamin, dietary supplement, mineral or combination thereof) having a second physical state (e.g., solid, liquid, gas or dispersion), wherein the physical state of the ingredient(s) in the second receiving chamber varies from the physical state of the ingredient(s) in the first receiving chamber. As contemplated and disclosed hereinabove, after the ingredients are introduced into the respective receiving chambers 528a, 528b, the cap 522 may be positioned in sealing relationship with the base 524 of the secondary capsule 520.

Still referring to FIG. 8, as illustrated by way of example, and not by way of restriction, a solid may be selectively introduced into at least a portion of the internal periphery of the receiving chamber 528a and a liquid may be selectively introduced into at least a portion of the internal periphery of the receiving chamber 528b. Although the ingredient(s) introduced into one of the receiving chamber 528a may be the same ingredient or may be different from the ingredient(s) introduced into receiving chamber 528b, the active ingredient(s) in the first receiving chamber 528a comprise a physical state (e.g., solid, liquid, gas or dispersion) that is different from the physical state of the active ingredient(s) in receiving chambers 528b. Those skilled in the art will readily recognize other possible modifications and adaptations relative to the contemplated variations in physical states (e.g., solid, liquid, gas and dispersion) of the active ingredient(s) selectively introduced within the receiving chambers 528 which are consistent with the spirit and scope of the present invention. It is intended, therefore, that the figures and examples provided herein be viewed as exemplary of the principles of the present invention, and not as restrictive to a particular structure or method for implementing those principles One presently preferred embodiment of an encapsulation process, as defined by the structural configuration of the multi-compartment capsule 510, may comprise the steps of: (1) introducing one or more active ingredients or medicaments (e.g., pharmaceutical, biotechnical, nutraceutical, vitamin, dietary supplement, mineral or combination thereof) into receiving chamber 518e defined by dividing walls 516d, 516e which are disposed vertically along the length of the elongated body of the base 514; (2) introducing a secondary capsule 520 into receiving chamber 518d defined by dividing walls 516c, 516d which are disposed vertically along the length of the elongated body of the base 514; (3) introducing one or more active ingredients or medicaments (e.g., pharmaceutical, biotechnical, nutraceutical, vitamin, dietary supplement, mineral or combination thereof) into receiving chamber 516c defined by dividing walls 516b, 516c which are disposed vertically along the length of the elongated body of the base 514; (4) introducing one or more active ingredients or medicaments (e.g., pharmaceutical, biotechnical, nutraceutical, vitamin, dietary supplement, mineral or combination thereof) into receiving chamber 518b defined by dividing walls 516b, 516e which are disposed vertically along the length of the elongated body of the base 514; (5) disposing dividing wall 516a along the length of the elongated body of the base 514 perpendicular to the disposition of dividing walls 516b, 516c, 516d, 516e and introducing one or more active ingredients or medicaments (e.g., pharmaceutical, biotechnical, nutraceutical, vitamin, dietary supplement, mineral or combination thereof) into receiving chamber 518a; and (6) selectively positioning the cap 512 in sealing relationship with the base 514 to form one presently preferred embodiment of a single, dosage multi-compartment capsule 510. As appreciated, the dividing wall 516a may also function in the formation of the sealing relationship between the base 514 and the corresponding cap 512, if structurally desired.

As illustrated by way of example, and not by way of limitation, a solid may be selectively introduced into at least a portion of the internal periphery of receiving chamber 518a, a dispersion may be selectively introduced into at least a portion of the internal periphery of receiving chamber 518b, a liquid may be selectively introduced into at least a portion of the internal periphery of the receiving chamber 516c and a liquid may be selectively introduced into at least a portion of the internal periphery of the receiving chamber 518e. Although the ingredient(s) introduced into one of the receiving chambers 518 may be the same ingredient or may be different from the ingredient(s) introduced into the other receiving chambers, the active ingredient(s) in at least two of the receiving chambers 518 preferably comprise a physical state (e.g., solid, liquid, gas or dispersion) that is different from the physical state of the active ingredient(s) in one or more of the remaining receiving chambers. Those skilled in the art will readily recognize other possible modifications and adaptations relative to the contemplated variations in physical states (e.g., solid, liquid, gas and dispersion) of the active ingredient(s) selectively introduced within the receiving chambers 518 which are consistent with the spirit and scope of the present invention. It is intended, therefore, that the figures and examples provided herein be viewed as exemplary of the principles of the present invention, and not as restrictive to a particular structure or method for implementing those principles.

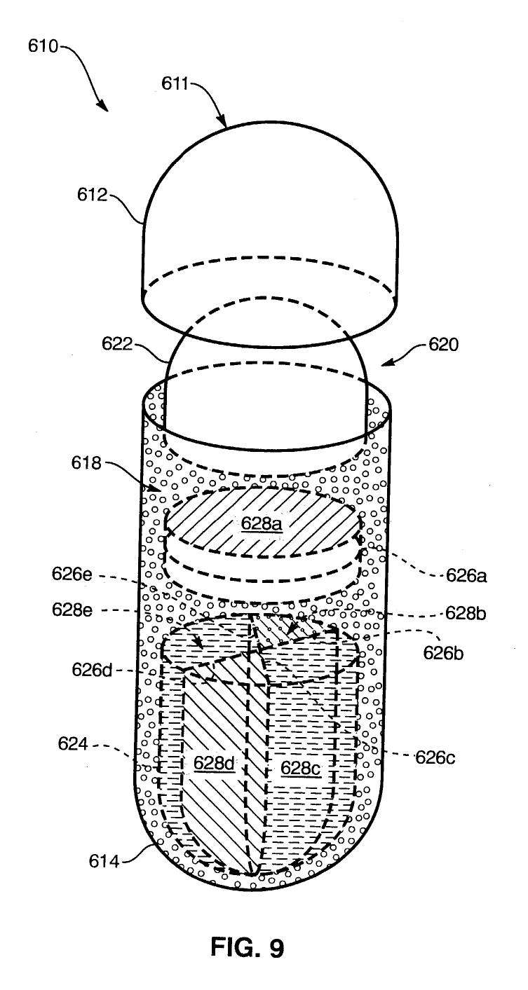

Referring now to FIG. 9, yet another presently preferred embodiment of a multi-compartment capsule of the present invention, generally designated as 610, is shown comprising a primary capsule 611 and a secondary capsule 620 selectively positionable within at least a portion of an internal periphery of the primary capsule. The primary capsule 611 having a receiving chamber 618 preferably formed having an internal periphery sufficient for receiving the secondary capsule 620, together with one or more active ingredients or medicaments (e.g., pharmaceutical, biotechnical, nutraceutical, vitamin, dietary supplement, mineral or combination thereof) therein. The secondary capsule 620 comprising a capsular base 624 preferably configured having an elongated or longitudinally extending body, a corresponding cap 622 and a plurality of dividing walls 626 selectively disposed along the length of the longitudinally extending body of the base, both horizontally and vertically. In structural design, the size, shape and positioning of the dividing walls 626a, 626b, 626c, 626d along the length of the longitudinally extending body of the base 624 facilitate the formation of a plurality of independent receiving chambers 628.

In one presently preferred embodiment, the dividing walls 626a, 626b, 626c, 626d, 426e are preferably disposed or seated in a spaced apart relationship within the internal periphery of the base 624 of the secondary capsule 620 along the length of the longitudinally extending body, whereby forming five (5) independent receiving chambers 628a, 628b, 628c, 628d, 628e. Each receiving chamber 628a, 628b, 628c, 628d, 628e of the secondary capsule 620 are preferably configured having an internal periphery dimensionally sufficient for receiving one or more active ingredients or medicaments (e.g., pharmaceutical, biotechnical, nutraceutical, vitamin, dietary supplement, mineral or combination thereof) therein.

One presently preferred embodiment of an encapsulation process, as defined by the structural configuration of the multi-compartment capsule 610, may comprise the steps of: (1) introducing one or more active ingredients or medicaments (e.g., pharmaceutical, biotechnical, nutraceutical, vitamin, dietary supplement, mineral or combination thereof) into receiving chamber 628e defined by dividing walls 626d, 626e which are vertically disposed along the length of the elongated body of the base 624; (2) introducing one or more active ingredients or medicaments (e.g., pharmaceutical, biotechnical, nutraceutical, vitamin, dietary supplement, mineral or combination thereof) into receiving chamber 628d defined by dividing walls 626c, 626d which are vertically disposed along the length of the elongated body of the base 624; (3) introducing one or more active ingredients or medicaments (e.g., pharmaceutical, biotechnical, nutraceutical, vitamin, dietary supplement, mineral or combination thereof) into receiving chamber 626c defined by dividing walls 626b, 626c which are vertically disposed along the length of the elongated body of the base 624; (4) introducing one or more active ingredients or medicaments (e.g., pharmaceutical, biotechnical, nutraceutical, vitamin, dietary supplement, mineral or combination thereof) into receiving chamber 628b defined by dividing walls 626b, 626e which are vertically disposed along the length of the elongated body of the base 624; (5) disposing dividing wall 626a along the length of the elongated body of the base 624 perpendicular to the disposition of dividing walls 626b, 626c, 626d, 626e and introducing one or more active ingredients or medicaments (e.g., pharmaceutical, biotechnical, nutraceutical, vitamin, dietary supplement, mineral or combination thereof) into receiving chamber 628a; (6) selectively positioning the cap 622 in sealing relationship with the base 624 of the secondary capsule 620; (7) introducing the secondary capsule 620 and one or more ingredients or medicaments (e.g., pharmaceutical, biotechnical, nutraceutical, vitamin, dietary supplement, mineral or combination thereof) into the receiving chamber 618 of the primary capsule 611; and (8) selectively positioning the cap 612 in sealing relationship with the base 614 of the primary capsule 611 to form one presently preferred embodiment of a single, dosage multi-compartment capsule 610.

As illustrated by way of example, and not by way of restriction, a solid may be selectively introduced into at least a portion of the internal periphery of the receiving chamber 628a, a dispersion may be selectively introduced into at least a portion of the internal periphery of the receiving chamber 628b, a liquid may be selectively introduced into at least a portion of the internal periphery of the receiving chamber 628c, a solid may be selectively introduced into at least a portion of the internal periphery of the receiving chamber 628d and a liquid may be selectively introduced into at least a portion of the internal periphery of the receiving chamber 628e of the secondary capsule 620. In addition, a gas may be introduced into at least a portion of the internal periphery of the receiving chamber 618 of the primary capsule 611.

Although the ingredient(s) introduced into one of the receiving chambers 618, 628 of the primary and secondary capsules, respectively, may be the same ingredient or may be different from the ingredient(s) introduced into the other receiving chambers, the active ingredient(s) in at least two of the receiving chambers 618, 628 preferably comprise a physical state (e.g., solid, liquid, gas or dispersion) that is different from the physical state of the active ingredient(s) in one or more of the remaining receiving chambers. Those skilled in the art will readily recognize other possible modifications and adaptations relative to the contemplated variations in physical states (e.g., solid, liquid, gas and dispersion) of the active ingredient(s) selectively introduced within the receiving chambers 618, 628 which are consistent with the spirit and scope of the present invention. It is intended, therefore, that the figures and examples provided herein be viewed as exemplary of the principles of the present invention, and not as restrictive to a particular structure or method for implementing those principles.

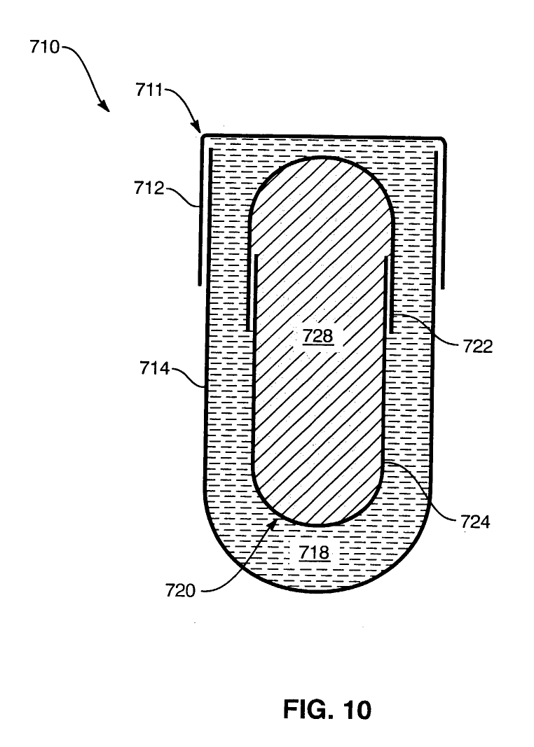

Another presently preferred embodiment of a multi-compartment capsule of the present invention, generally designated as 710 in FIG. 10, is shown comprising a secondary capsule 720 including one or more active ingredients or medicaments (e.g., pharmaceutical, biotechnical, nutraceutical, vitamin, dietary supplement, mineral or combination thereof) within at least a portion of the internal periphery of a receiving chamber 728 and having a size and shape sufficient for being selectively introduced within at least a portion of the internal periphery of a receiving chamber 718 of a primary capsule 711. The primary capsule 711 also includes one or more active ingredients or medicaments (e.g., pharmaceutical, biotechnical, nutraceutical, vitamin, dietary supplement, mineral or combination thereof) introduced within the internal periphery of the receiving chamber 718, wherein the active ingredient(s) introduced into the primary capsule comprises a physical state (e.g., solid, liquid, gas or dispersion) which differs from the physical state of the active ingredient(s) introduced into the internal periphery of the secondary capsule. In structural design, the primary capsule 711 further comprises a cap 712 having a generally U-shaped configuration adapted to provide a sealing relationship when engaging the corresponding base 714, thereby reducing dead space volume in the internal periphery of the receiving chamber 718 of the base. In this regard, the configuration of the cap 712 generally eliminates or substantially reduces the potential dead space volume within the internal periphery of the receiving chamber 718, thus functionally negating the opportunity for reaction between an air bubble and the active ingredient(s) introduced into the base 714 of the primary capsule 711.

One presently preferred embodiment of an encapsulation process, as defined by the structural configuration of the multi-compartment capsule 710, may include the steps of: (1) introducing one or more active ingredients or medicaments (e.g., pharmaceutical, biotechnical, nutraceutical, vitamin, dietary supplement, mineral or combination thereof) into receiving chamber 728; (2) selectively positioning the cap 722 in sealing relationship with the base 724 of the secondary capsule 720; (3) introducing one or more active ingredients or medicaments (e.g., pharmaceutical, biotechnical, nutraceutical, vitamin, dietary supplement, mineral or combination thereof), together with the secondary capsule 720, into the receiving chamber 718 of the primary capsule 711; and (4) selectively positioning the cap 712 having a general U-shaped configuration in sealing relationship with the base 714 of the primary capsule 711 to form a presently preferred embodiment of a single, dosage multi-compartment capsule 710, wherein eliminating or substantially reducing dead space volume within the internal periphery of the receiving chamber 718.

A solid is selectively introduced within at least a portion of the internal periphery of the receiving chamber 728 of the secondary capsule 720 and a liquid is selectively introduced within at least a portion of the internal periphery of the receiving chamber 718 of the primary capsule 711. Although the ingredient(s) introduced into the receiving chamber 718 of the primary capsule 711 may be the same or different from the ingredient(s) introduced into the receiving chamber 728 of the secondary capsule 720, the active ingredient(s) in the primary capsule have a physical state (i.e., solid, liquid, gas or dispersion) that various from the physical state of the active ingredient(s) in the secondary capsule. Accordingly, those skilled in the art will readily recognize other possible modifications and adaptations relative to the contemplated variations in physical states of the active ingredient(s) selectively introduced within the receiving chambers 718, 728 of the primary and secondary capsules 711, 720, respectively, which are consistent with the spirit and scope of the present invention. It is intended, therefore, that the figures and examples provided herein be viewed as exemplary of the principles of the present invention, and not as restrictive to a particular structure or method for implementing those principles.

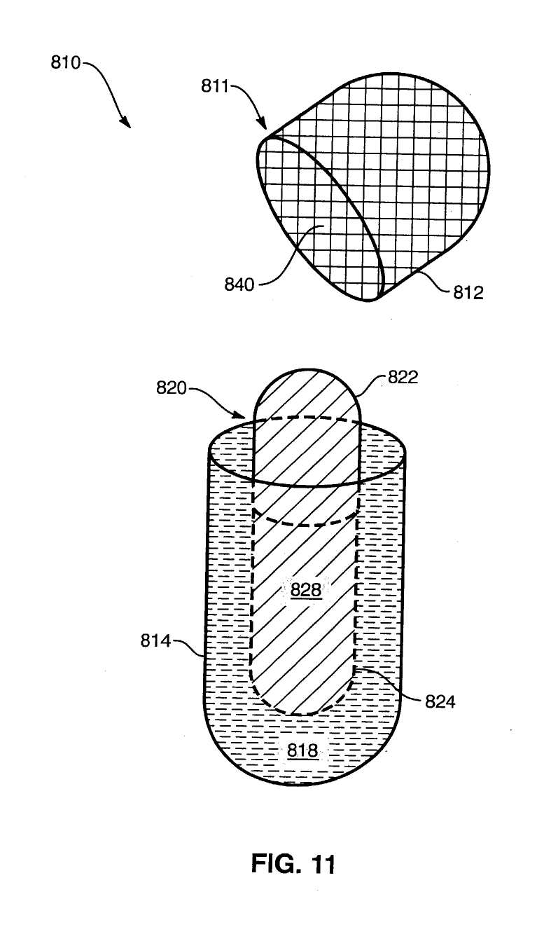

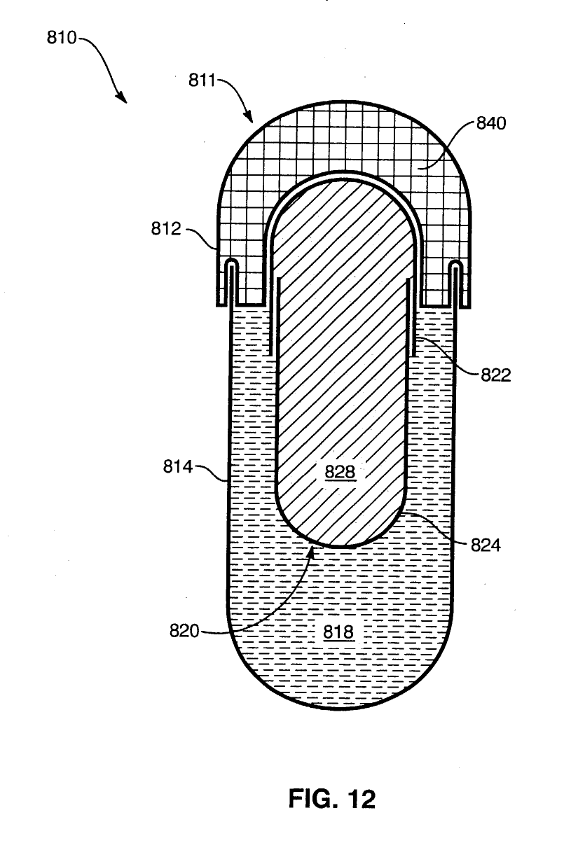

Referring now to FIGS. 11 and 12, yet another presently preferred embodiment of a multi-compartment capsule 810 of the present invention is shown comprising a secondary capsule 820 including one or more active ingredients or medicaments (e.g., pharmaceutical, biotechnical, nutraceutical, vitamin, dietary supplement, mineral or combination thereof) within at least a portion of the internal periphery of a receiving chamber 828. The secondary capsule 820 being preferably formed having a size and shape sufficient for being selectively introduced within at least a portion of the internal periphery of a receiving chamber 818 of a primary capsule 811. Similarly, the primary capsule 811 includes one or more active ingredients or medicaments (e.g., pharmaceutical, biotechnical, nutraceutical, vitamin, dietary supplement, mineral or combination thereof) introduced within the internal periphery of the receiving chamber 818, together with the secondary capsule 820, wherein the active ingredient(s) introduced into the primary capsule comprises a physical state (e.g., solid, liquid, gas or dispersion) which differs from the physical state of the active ingredient(s) introduced into the internal periphery of the secondary capsule 820.

In preferred structural design, the primary capsule 811 comprises a cap 812 having a general cylindrical configuration adapted to provide a sealing relationship when engaging the corresponding base 814 to form a single dosage, multi-compartment capsule 810. An amount of filling material 840 may be introduced into the internal periphery of the cap 812 to fill, either partially or completely, the inner volume of the cap, thereby reducing the dead space volume in the internal periphery of the receiving chamber 818 of the base. In this regard, the configuration of the addition of the filler material 840 relative to the internal periphery of the cap 812 generally eliminates or substantially reduces the potential dead space volume within the internal periphery of the receiving chamber 818, thus functionally negating the potential for a reaction between an air bubble and the active ingredient(s) introduced into the base 814 of the primary capsule 811.

Preferably, the filling material 840 introduced into at least a portion of the internal periphery of the cap 812 may include a hydrophilic polymer, such as gelatin. It will be readily appreciated by those skilled in the art that other filling materials may be used, such as, for example, starch, casein, chitosan, soya bean protein, safflower protein, alginates, gellan gum, carrageenan, xanthan gum, phtalated gelatin, succinated gelatin, cellulosephtalate-acetate, polyvinylacetate, hydroxypropyl methyl cellulose (HPMC), oleoresin, polyvinylacetate-phtalate, polymerisates of acrylic or methacrylic esters, or combinations thereof. In other presently preferred embodiments of the present invention, the filling material 840 may include the introduction of an inert compound, for example, nitrogen gas into at least a portion of the internal periphery of the cap 811. Based on the principals of eliminating or reducing the volume dead space in multi-compartment capsules disclosed herein, those skilled in the art will readily recognize other possible modifications and combinations which are consistent with the spirit and scope of the present invention. It is intended, therefore, that the examples provided herein be viewed as exemplary of the principles of the present invention, and not as restrictive to a particular structure or process for implementing those principles.

The filling material 840 introduced within at least a portion of the internal periphery of the cap 812 of the primary capsule 811 is generally intended to promote a binding contact with at least a portion of the cap 822 of the secondary capsule 820, thereby seating at least a portion of the secondary capsule within the cap of the primary capsule and forming a molded appearance. As appreciated, the introduction of the filling material 840 into the cap 812 of the primary capsule 811 prior to the joining and sealing process may prevent the opportunity for a reaction between an air bubble and the active medicament(s) within the receiving chamber 818 of the primary capsule, while preserving the overall rounded shape of the multi-compartment capsule 910 for ease of swallowing by a consumer.

As best illustrated in FIG. 12, one presently preferred embodiment of an encapsulation process, as defined by the structural configuration of the multi-compartment capsule 810, may include the steps of: (1) introducing one or more active ingredients or medicaments (e.g., pharmaceutical, biotechnical, nutraceutical, vitamin, dietary supplement, mineral or combination thereof) into at least. a portion of the receiving chamber 828; (2) selectively positioning the cap 822 in sealing relationship with the base 824 of the secondary capsule 820; (3) introducing one or more active ingredients or medicaments (e.g., pharmaceutical, biotechnical, nutraceutical, vitamin, dietary supplement, mineral or combination thereof), together with the secondary capsule 820, into at least a portion of the receiving chamber 818 of the primary capsule 811; (4) introducing a filling material 840 into at least a portion of the internal periphery of the cap 812 (i.e., filling the cap); and (5) selectively positioning the cap 812 having a general conical configuration in sealing relationship with the base 814 of the primary capsule 811 to form one presently preferred embodiment of a single, dosage multi-compartment capsule 810, wherein eliminating or substantially reducing dead space volume within the internal periphery of the cap 812 and the receiving chamber 818, respectively.

A solid may be selectively introduced within at least a portion of the internal periphery of the receiving chamber 828 of the secondary capsule 820 and a liquid may be selectively introduced within at least a portion of the internal periphery of the receiving chamber 818 of the primary capsule 811. Although the ingredient(s) introduced into the receiving chamber 818 of the primary capsule 811 may be the same or different from the ingredient(s) introduced into the receiving chamber 828 of the secondary capsule 820, the active ingredient(s) in the primary capsule have a physical state (i.e., solid, liquid, gas or dispersion) that various from the physical state of the active ingredient(s) in the secondary capsule. Accordingly, those skilled in the art will readily recognize other possible modifications and adaptations relative to the contemplated variations in physical states of the active ingredient(s) selectively introduced within the receiving chambers 818, 828 of the primary and secondary capsules 811, 820, respectively, which are consistent with the spirit and scope of the present invention. It is intended, therefore, that the figures and examples provided herein be viewed as exemplary of the principles of the present invention, and not as restrictive to a particular structure or method for implementing those principles.

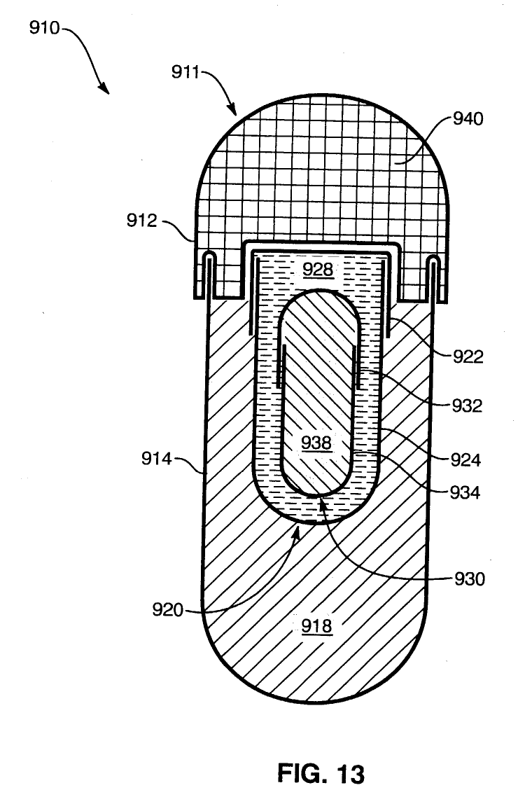

Referring now to FIG. 13, another presently preferred embodiment of a multi-compartment capsule, generally designated at 910, is shown comprising a tertiary capsule 930 including one or more active ingredients or medicaments (e.g., pharmaceutical, biotechnical, nutraceutical, vitamin, dietary supplement, mineral or combination thereof) within at least a portion of the internal periphery of a receiving chamber 938 and having a size and shape sufficient for being introduced into the internal periphery of a receiving chamber 928 of a secondary capsule 920. The secondary capsule 920 having one or more active ingredients or medicaments (e.g., pharmaceutical, biotechnical, nutraceutical, vitamin, dietary supplement, mineral or combination thereof) introduced within at least a portion of the internal periphery of a receiving chamber 928, together with the tertiary capsule 930. The secondary capsule 920 preferably formed having a size and shape sufficient for being selectively introduced within at least a portion of the internal periphery of a receiving chamber 918 of a primary capsule 911. Similarly, the primary capsule 911 may include one or more active ingredients or medicaments (e.g., pharmaceutical, biotechnical, nutraceutical, vitamin, dietary supplement, mineral or combination thereof) introduced within the internal periphery of the receiving chamber 818, together with the secondary capsule 920 which houses the tertiary capsule 930. In one presently preferred embodiment, the active ingredient(s) introduced into the secondary capsule 920 comprises a physical state (e.g., solid, liquid, gas or dispersion) which differs from the physical state of the active ingredient(s) introduced into the internal periphery of the primary capsule 911 and the internal periphery of the tertiary capsule 930.

In preferred structural design, the primary capsule 911 comprises a cap 912 having a general cylindrical configuration adapted to provide a sealing relationship when engaging the corresponding base 914 to form a single dosage, multi-compartment capsule 910. An amount of filling material 940 may be introduced into at least a portion of the internal periphery of the cap 912 to fill, either partially or completely, the inner volume of the cap, thereby reducing the dead space volume in the cap and the internal periphery of the receiving chamber 918 of the base. In this regard, the configuration of the addition of the filler material 940 relative to the internal periphery of the cap 912 may generally eliminate or substantially reduce the potential dead space volume within the internal periphery of the receiving chamber 918, thus functionally negating the potential for a reaction between an air bubble and the active ingredient(s) introduced into the base 914 of the primary capsule 911.

Preferably, the filling material 940 introduced into at least a portion of the internal periphery of the cap 912 may include a hydrophilic polymer, such as gelatin. It will be readily appreciated by those skilled in the art that other filling materials may be used, such as, for example, starch, casein, chitosan, soya bean protein, safflower protein, alginates, gellan gum, carrageenan, xanthan gum, phtalated gelatin, succinated gelatin, cellulosephtalate-acetate, polyvinylacetate, hydroxypropyl methylcellulose (HPMC), oleoresin, polyvinylacetate-phtalate, polymerisates of acrylic or methacrylic esters, or combinations thereof. In other presently preferred embodiments of the present invention, the filling material 840 may include the introduction of an inert compound, for example, nitrogen gas into at least a portion of the internal periphery of the cap 912. Based on the principals of eliminating or reducing the volume dead space in multi-compartment capsules disclosed herein, those skilled in the art will readily recognize other possible modifications and combinations which are consistent with the spirit and scope of the present invention. It is intended, therefore, that the examples provided herein be viewed as exemplary of the principles of the present invention, and not as restrictive to a particular structure or process for implementing those principles.

The filling material 940 introduced within at least a portion of the internal periphery of the cap 912 of the primary capsule 911 is generally intended to promote a binding contact with at least a portion of the cap 922 of the secondary capsule 920, thereby seating at least a portion of the secondary capsule within the cap of the primary capsule and forming a molded appearance. As appreciated, the introduction of the filling material 940 into the cap 912 of the primary capsule 911 prior to the joining and sealing process tends to prevent the opportunity for a reaction between an air bubble and the active medicament(s) within the receiving chamber 918 of the primary capsule, while preserving the overall rounded shape of the multi-compartment capsule 910 for ease of swallowing by a consumer.

As best illustrated in FIG. 13, one presently preferred embodiment of an encapsulation process, as defined by the structural configuration of the multi-compartment capsule 910, may include the steps of: (1) introducing one or more active ingredients or medicaments (e.g., pharmaceutical, biotechnical, nutraceutical, vitamin, dietary supplement, mineral or combination thereof) into at least a portion of the receiving chamber 938 of a tertiary capsule 930; (2) selectively positioning the cap 932 in sealing relationship with the base 934 of the tertiary capsule 930; (3) introducing one or more active ingredients or medicaments (e.g., pharmaceutical, biotechnical, nutraceutical, vitamin, dietary supplement, mineral or combination thereof), together with the tertiary capsule 930, into at least a portion of the receiving chamber 928 of the secondary capsule 920; (4) selectively positioning the cap 922 in sealing relationship with the base 924 of the secondary capsule 920; (5) introducing one or more active ingredients or medicaments (e.g. pharmaceutical, biotechnical, nutraceutical, vitamin, dietary supplement, mineral or combination thereof), together with the secondary capsule 920, into at least a portion of the receiving chamber 918 of the primary capsule 911; (6) introducing a filling material 940 into at least a portion of the internal periphery of the cap 912 (i.e., preferably filling the cap); and (7) selectively positioning the cap 912 having a general conical configuration in seating relationship with at least a portion of the secondary capsule 920 and sealing the base 914 of the primary capsule 911 to form one presently preferred embodiment of a single, dosage multi-compartment capsule 910, wherein eliminating or substantially reducing dead space volume within the internal periphery of the cap 912 and the receiving chamber 918, respectively.

A solid may be introduced within at least a portion of the internal periphery of the receiving chamber 938 of the tertiary capsule 930, a liquid may be introduced into at least a portion of the internal periphery of the secondary capsule 920 and a solid may be selectively introduced within at least a portion of the internal periphery of the receiving chamber 918 of the primary capsule 911. Although the ingredient(s) introduced into the receiving chambers 918, 928, 938 of the primary, secondary and tertiary capsules 911, 920, 930, respectively, may be the same or different from the ingredient(s) introduced into the other receiving chambers, the active ingredient(s) in at least two of the receiving chambers 918, 928, 938 have different physical states (i.e., solid, liquid, gas or dispersion). Those skilled in the art will readily recognize other possible modifications and adaptations relative to the contemplated variations in physical states of the active ingredient(s) selectively introduced within the receiving chambers 918, 928, 938 of the primary, secondary and tertiary capsules 911, 920, 930, respectively, which are consistent with the spirit and scope of the present invention. It is intended, therefore, that the figures and examples provided herein be viewed as exemplary of the principles of the present invention, and not as restrictive to a particular structure or method for implementing those principles.

Generally referring to FIGS. 1-13, the component parts of the presently preferred embodiments of the multi-compartment capsules (i.e., capsular base, corresponding cap and dividing walls) of the present invention may comprise a hydrophilic polymer, such as gelatin (marine or animal based product). Other suitable materials forming the capsules may include starch, casein, chitosan, soya bean protein, safflower protein, alginates, gellan gum, carrageenan, xanthan gum, phtalated gelatin, succinated gelatin, cellulosephtalate-acetate, polyvinylacetate, hydroxypropyl methyl cellulose (HPMC), oleoresin, polyvinylacetate-phtalate, polymerisates of acrylic or methacrylic esters, or combinations thereof. The material comprising the capsular components may further include between about 0% to 40% of pharmaceutically acceptable plasticizers based upon the weight of the hydrophilic polymer. Plasticizers that may be employed include, for example and not by way of limitation, polyethylene glycol, glycerol, sorbitol, dioctyl-sodium sulfosuccinate, triethyl citrate, tributyl citrate, 1,2-propyleneglycol, mono-acetates, di-acetates, or tri-acetates of glycerol, mixtures thereof, or the like. As appreciated, plasticizers may also be used in the development of a soft elastic shell, often referred to as a soft gelatin capsule or “soft gel” capsule, for a primary capsule, a secondary capsule and/or a tertiary capsule.

The capsular shell material may contain pharmaceutically acceptable lubricants in the range of about 0% to 10%, based upon the weight of the hydrophilic polymer. Lubricants that may be used include, for example and not by way of limitation, aluminum stearate, calcium stearate, magnesium stearate, tin stearate, talc, sodium lauryl sulfate, lecithins, mineral oils, stearic acid, silicones, mixtures thereof, or the like. One presently preferred embodiment of the multi-compartmental capsules of the present invention (e.g., primary capsule, secondary capsule, tertiary capsule, etc.) may include, for example, LICAPS® capsules (for poorly soluble compounds), VCAPSTM capsules (made from cellulosic raw materials), CONI-SNAP® capsules and PRESS-FIT® capsules which are all presently manufactured by Capsugel, a subsidiary of Pfizer, Inc.

In one presently preferred embodiment of an encapsulation process, the primary capsule may be kept under conditions of low humidity within a filling machine during the contemplated steps of rectifying and assembling. In certain embodiments, the primary capsule may contain moisture content in the range of approximately 0% to 6% of the total weight. Similarly, a secondary capsule, a tertiary capsule, etc. may be processed in the same manner as the primary capsule relative to conditions of low humidity during the steps of rectifying and assembling. As contemplated herein, a moisture content of approximately 0% to 3% by weight is preferable. However, capsules having a higher moisture content than those stated herein are certainly not outside the spirit and scope of the present invention.

As illustrated in FIGS. 1-9 and 11-13, the shape of the base and corresponding cap of the capsules (e.g., primary, secondary, tertiary, etc.) of the presently preferred embodiments of the multi-compartment capsules are configured having a general cylindrical shape which defines a diameter and length sufficient for the introduction of an internal smaller capsule or one or more dividing walls along the length of the capsular base. It is apparent that other geometrical configurations of the cap are likewise suitable and contemplated herein, such as the general U-shaped configuration of the cap shown in FIG. 10. It is intended, therefore, that the examples provided herein be viewed as exemplary of the principles of the present invention, and not as restrictive to any particular structure or configuration for implementing those principles.

In one presently preferred embodiment, the clearance between the primary capsule and the secondary capsule introduced within the internal periphery of the primary capsule is preferably greater than +0.2 mm. The clearance between the outer capsular walls of the secondary capsule and the inner capsular walls of the primary capsule (or the tertiary capsule and the secondary capsule) may be in the range of about 0 mm to 0.5 mm, whereas the outer capsular walls of the secondary capsule or tertiary capsule may be in actual contact with the inner capsular walls of the primary capsule or secondary capsule, respectively. As appreciated, in an effort to structural facilitate independent receiving chambers on opposing sides of a dividing wall introduced within the internal periphery of a base of a capsule, the perimeter of the dividing wall preferably engages the inner capsular walls of the capsule to provide a sealing relationship therebetween.

As further contemplated herein, the inner capsular walls of a primary capsule may be treated with an adhesive sufficient to improve engagement between the primary capsule and the outer capsular walls of a secondary capsule. A suitable technique to apply an adhesive may be by way of spraying the same on the shells and capsules immediately before assembling the same. Suitable adhesives that may be used may include, for example, tackidex, an aqueous gelatin solution, or the like.

The primary, secondary or tertiary capsules, in accordance with the present invention, may be formed having the same or different colors. Moreover, the base and corresponding cap of a single capsule may be formed having different colors in an effort to enhance the aesthetics of the capsule to the consumer. In one presently preferred embodiment of a multi-compartment capsule of the present invention, the dosage may be banded, sealed or easily dividable in a contact area of the primary and secondary capsules or the sealing band may be color-coded to assist in branding, if desired.

It is further contemplated herein that a multi-compartment capsule of the present invention may comprise component parts of the capsule having various time-release coatings to facilitate the release and ultimately the absorption of those active ingredients introduced into the different receiving chambers of the multi-compartment capsule to release at different release rates. In particular, a primary capsule may be formed having a conventional time-release coating that dissolves and releases the active ingredient(s) contained therein before the timed-release of the active ingredient(s) contained within a secondary capsule. Likewise, the dividing walls disposed within the internal periphery of the base of a capsule may be formed having conventional time-release coatings that dissolve and release the active ingredients within each receiving chamber defined by the dividing walls at different rates, thereby delivering the active ingredients or medicaments contained within a multi-compartment capsule at different rates. Certain active ingredients or medicaments may, therefore, be delivered at a selected interval, while other ingredients may be released at a later interval. In this way, the novel design of the multi-compartment capsules of the present invention may facilitate precision delivery of active ingredients to targeted areas of the consumer.

The disclosure of secondary and tertiary capsules may be replaced with other forms of microencapsulation. Microencapsulation, as previously described, refers to the process whereby minute parcels of a solid, liquid, gas or dispersion, introduced into one or more of the receiving chambers as active ingredient(s), are film-coated with a secondary material in order to shield the active ingredient from its surrounding environment. Microcapsules may measure from microns to several millimeters, whereas the main purpose being to facilitate the release of the active ingredients at different release rates.

The incorporation of time-release coatings to varying the release rates of the active ingredients of a multi-compartment capsule may be used to target key time intervals or events when the body may be most able to utilize the active ingredients. In one presently preferred embodiment of the present invention, all of the active ingredients may be microencapsulated. In alternate presently preferred embodiments, only selected ingredients may be microencapsulated for delayed release, while other ingredients may be provided for immediate absorption. Thus, the incorporation of time-release coatings in the encapsulation process when forming a multi-compartment capsule may be specifically designed to fit the needs and desires of numerous different users having similar conditions that are being targeted for treatment.

As contemplated herein, the physical states of active ingredients are characterized into one of four different states (e.g., solid, liquid, gas or dispersion). These four different states are sometimes referred to as “phases” (i.e., solid phase, liquid phase, gas phase or dispersion phase). For purposes of the present invention, the term “solid” is defined as including, by way of example only and not by way of limitation, pills, tablets, capsules (including both hard and soft elastic), powders, granulation, flakes, troches (lozenges and pastilles), suppositories and semi-solid pastes, ointments, emulsions or creams. The term “liquid” is defined as including, by way of example only and not by way of limitation, solutions, spirits, elixirs or fluid extracts. The term “dispersion” is defined as including, by way of example only and not by way of limitation, aerosols (liquid or solid in gas), suspensions (solid in liquid), emulsions (liquid in liquid), foams (gas in liquid), solid foams (solid in gas) or gels (liquid or solid in solid).

The active ingredients or medicaments introduced into the receiving chambers of the multi-compartment capsules of the present invention preferably comprise a pharmaceutical, biotechnical, nutraceutical, vitamin, dietary supplement, mineral or combination thereof. For purposes of the present invention, the term “pharmaceutical” is defined as any substance that affects the structure or functioning of a living organism. Pharmaceuticals, sometimes referred to as “drugs” are widely used for the prevention, diagnosis and treatment of diseases and for the relief of symptoms. The term “biotechnical” is defined as any substance that is derived from a biotechnology process. Biotechnology, sometimes shortened to “biotech”, is the development of techniques (e.g., genetic engineering, protein engineering, genomics, proteomics, monoclonal antibody production, polymerase chain reaction and the like) for the application of biological processes to the production of materials of use in medicine and industry. The term “nutraceutical” is defined as any substance that is a food of a part of a food and provides medical or health benefits, including the prevention and treatment of disease. The term “vitamin” is defined as any of various organic substances or compounds that are essential for the normal processes of growth and maintenance (e.g., essential for energy transformation and regulation of metabolism) of the body which are present in natural foodstuffs or sometimes produced within the body. The term “dietary supplement” is defined as any product (other than tobacco) intended to supplement the diet that bears or contains one or more of the following dietary ingredients: (A) a vitamin; (B) a mineral; (C) an herb or other botanical; (D) an amino acid; (E) a dietary substance for supplementing the diet by increasing the total dietary intake; or (F) a concentrate, metabolite, constituent, extract or combination of any ingredient described in (A), (B), (C), (D), or (E) hereinabove. If desired, excipients may also be introduced into one or more of the receiving chambers of the multi-compartment capsules of the present invention in addition to the active ingredient(s). For example, in some cases involving active ingredients or medicaments with poor water solubility, it may be desirous to stabilize the liquids, solids or dispersions using a lipid, lipoid, lecithin, ghee or the like.

The following examples will illustrate the invention in further detail. It will be readily understood that the various active ingredients or medicaments that may be introduced into the receiving chambers of the multi-compartment capsules of the present invention, as generally described and illustrated in the Examples herein, are to be viewed as exemplary of the principles of the present invention, and not as restrictive to a particular structure or process for implementing those principles. Thus, the following more detailed description of the presently preferred embodiments of the methods, formulations, and compositions of the present invention, as represented in Examples I-IV, is not intended to limit the scope of the invention, as claimed, but is merely representative of presently preferred embodiments of the invention.

### EXAMPLE I

### Fluoxetine (solid), S-adenosylmethione (SAMe) (solid) & Vitamin E (liquid)

As appreciated by those skilled in the art, depression is a mental state characterized by excessive sadness. Depression is one of several forms of mood disorders. Activity in those affected with depression may be agitated and restless or slow and retarded. Those affected may also show pessimistic or despairing behavior and may have disturbances in sleep, appetite and concentration. Depression is often a co-morbid condition with other chronic disease states involving the neurological system, cardiovascular system, respiratory system, endocrine system, musculoskeletal system, immune system, genitourinary system and the like. This list is should not be considered exclusive.

Administration of Fluoxetine is known by those of skill in the art to alleviate the signs and symptoms of depression. Fluoxetine belongs to a class of compounds which are given the functional name: selective serotonin re-uptake inhibitors (SSRI's). This class may include, for example: fluoxetine (PROZAC®), sertraline (ZOLOFT®), paroxetine (PAXIL®), fluvoxamine (LUVOX®), citalopram (CELEXA®) and escitalopram (LEXAPRO®). As appreciated, the foregoing list is provided herein as exemplary and should not be considered exclusive or exhaustive.

Fluoxetine is a bicyclic compound, similar in structure to phenylpropanolamine. Fluoxetine structure imparts a high selectivity for interaction with cells of the nervous system for the function of preventing the re-uptake of serotonin into pre-synaptic cell storage sites. This action leads to marked increases in synaptic concentration of serotonin and is facilitative of numerous physiological processes requiring serotonin neurotransmission. In the pharmaceutical field Fluoxetine is available as a hydrochloride salt (HCl).

S-adenosylmethione (SAMe), is derived from two materials: methionine, a sulfur-containing amino acid, and adenosine triphosphate (ATP), the body's main energy compound. SAMe was originally developed around 1950 as an antidepressant, but it was also found to be helpful in the alleviation of arthritic symptoms. SAMe is essential for the manufacture of melatonin, which is needed to regulate sleep. It also helps to protect DNA from harmful mutations and may help prevent certain types of nerve damage. Current clinical research is beginning to confirm these antidepressant qualities of SAMe.

Vitamin E, also named alpha-tocopherol, is a well-known scavenger of free-radicals in the body. Free-radical scavengers are sometimes referred to as anti-oxidants. This scavenging process is important for detoxifying the body of chemicals which are known to promote apoptosis, or programmed cell death. Apoptosis is a scientific description of cellular destruction. Although it is a popular anti-oxidant, Vitamin E is poorly soluble in water and thus can be administered only as a liquid-oil formulation or in an oil formulation enclosed in a soft elastic capsule. Vitamin E is typically measured in international units (IU) of alpha tocopherol.

In one presently preferred embodiment of the present invention, therapeutically effective amounts of Fluoxetine, SAMe and Vitamin E (active ingredients) may be introduced into receiving chambers of a multi-compartment capsule wherein Fluoxetine and SAMe comprises a physical state (e.g., solid, liquid, gas or dispersion) different from the physical state of Vitamin E. As shown in FIGS. 3 and 4, a therapeutically effective amount of Fluoxetine and SAMe may be introduced into receiving chamber 218a and a therapeutically effective amount of Vitamin E may be introduced into receiving chamber 218b of a multi-compartment capsule 210 of the present invention. Consistent with the foregoing, multi-compartment, multi-phase capsules and encapsulation technology are herein contemplated to produce a delivery vehicle for delivering mood enhancing, anti-depressant and anti-oxidant compounds to the body in a single dosage. A capsular format of the present invention may include the following composition:

| | |
|---|---|
| Receiving Chamber (218a): | |
| Fluoxetine | 20 mg |
| [20-60 mg/day] | |
| S-adenosylmethione | 1000 mg |
| [200-1600 mg/day] | |
| Receiving Chamber (218b): | |
| Vitamin E | 200 IU |
| [200-400 IU/day] | |

The incorporation of time-release coatings to varying the release rates of the active ingredients (e.g., Fluoxetine/SAMe and Vitamin E) in the primary and secondary capsules, respectively, of the multi-compartment capsule may be used to target key time intervals or events when the body may be most able to utilize the named active ingredients. Thus, the incorporation of time-release coatings in the encapsulation process when forming a multi-compartment capsule may be specifically designed to fit the needs and desires of numerous different users having similar conditions that are being targeted for treatment.

According to one presently preferred embodiment of the present invention, a therapeutically effective amount of Fluoxetine and SAMe may be introduced into at least a portion of the receiving chamber 218a in the form of a solid and a therapeutically effective amount of Vitamin E may be introduced into at least a portion of the receiving chamber 218b of the primary capsule 211 in the form of a liquid.

In an alternative presently preferred embodiment of the present invention, therapeutically effective amounts of Fluoxetine and SAMe and Vitamin E (active ingredients) may be introduced into receiving chambers of a multi-compartment capsule wherein Fluoxetine and SAMe comprises a physical state (e.g., solid, liquid, gas or dispersion) different from the physical state of Vitamin E. As shown in FIG. 2, a therapeutically effective amount of Fluoxetine and SAMe, in the form of a solid, may be introduced into receiving chamber 118 and 138 and a therapeutically effective amount of Vitamin E, in the form of a liquid, may be introduced into receiving chamber 128 of a multi-compartment capsule 110 of the present invention. The material forming the primary capsule shell 111 may be formulated in a manner allowing for immediate dissolution and release of the of the contents of receiving chamber 118. The material forming the secondary capsule shell 120 may be formulated in a manner allowing for either an immediate dissolution or a time-delayed dissolution and release of the contents of receiving chamber 128. The material forming the tertiary capsule shell 138 may be formulated in a manner allowing for time-delayed dissolution and release of the contents of receiving chamber 138. In this presently preferred embodiment of the present invention, a total daily dosage of Fluoxetine and SAMe may be delivered as two separate dosages within a single oral dosage form. One presently preferred embodiment of the present invention thus makes for a more convenient dosage form.

Since the encapsulation process and multi-compartment, multi-phase capsule of the present invention are configured to apply to an anticipated treatment regime or medicinal design of a single dosage capsule, it will be readily appreciated that the introduction of one or more active ingredients into the receiving chambers of the primary and secondary capsules, respectively, is anticipated such that the various ingredients may be introduced in different receiving chambers to accommodate different treatment modalities. For example, a multi-compartment capsule may be formulated having Fluoxetine and SAMe introduced into the receiving chambers of the secondary capsule and Vitamin E may be introduced into the receiving chamber of the primary capsule. It is intended, therefore, that the examples provided herein be viewed as exemplary of the principles of the present invention, and not as restrictive to a particular structure or method for implementing those principles.

### EXAMPLE II

### Rofecoxib (solid) & Vitamin E (liquid)

As appreciated by those skilled in the art, arthritis is an inflammatory condition typically affecting the synovia and cartilage of joints. It has been estimated that as many as one in three persons may experience symptoms associated with arthritis during their lifetime.

In addition to arthritis, various other chronic, debilitating conditions may afflict the aged. Many of these conditions result from the natural process of aging in humans. The natural aging process is partially due to the accumulation and effects of toxic free-radical chemicals. Free-radicals result from several homeostatic biochemical processes. It is, accordingly, desirable to develop pharmaceutical, biotechnical, nutraceutical or dietary supplement products which may alleviate multiple chronic, debilitating conditions. It is also desirable to package and administer such products in the most economic and convenient possible fashion.

Anti-inflammatory agents may have many diverse therapeutic roles in the human body. Inflammation is the process undertaken by the body as it responds to an injury. A typical inflammatory response involves blood vessel dilation, increased blood flow to the site of injury, and influx of white blood cells to process and remove dead tissue. Inflammation can lead to pain and swelling at the site of injury. Medicaments used in modulating the inflammatory response may be divided into steroid and non-steroidal labels. The latter is more commonly identified as non-steroidal anti-inflammatory drugs (NSAIDs),.

Rofecoxib belongs to a class of NSAID compounds given the functional name cyclo-oxygenase-2 (“COX-2”) inhibitors. This class may include, for example: rofecoxib (VIOXX®), celecoxib (CELEBREX®), valdecoxib (BEXTRA®), and meloxicam (MOBIC®). As appreciated, the foregoing list is provided herein as exemplary and should not be considered exclusive or exhaustive.

Rofecoxib is presently believed to inhibit the action of COX-2, an enzyme involved in the production of prostaglandins in the human body. Prostaglandins serve many diverse roles, one of which is to stimulate an inflammation mechanism in immune responses. Recently, Rofecoxib was labeled for use in the treatment of osteoarthritis, rheumatoid arthritis, acute pain, and primary dysmenorrhea.

Vitamin E, also named alpha-tocopherol, is a well-known scavenger of free-radicals in the body. Free-radical scavengers are sometimes referred to as anti-oxidants. This scavenging process is important for detoxifying the body of chemicals which are known to promote apoptosis, or programmed cell death. Apoptosis is a scientific description of cellular destruction. Although it is a popular anti-oxidant, Vitamin E is poorly soluble in water and thus can be administered only as a liquid-oil formulation or in an oil formulation enclosed in a soft elastic capsule.

In one presently preferred embodiment of the present invention, therapeutically effective amounts of Rofecoxib and Vitamin E (active ingredients) may be introduced into receiving chambers of a multi-compartment capsule wherein Rofecoxib comprises a physical state (e.g., solid, liquid, gas or dispersion) different from the physical state of Vitamin E. As shown in FIGS. 3 and 4, a therapeutically effective amount of Rofecoxib may be introduced into receiving chamber 218a and a therapeutically effective amount of Vitamin E may be introduced into receiving chamber 218b of a multi-compartment capsule 210 of the present invention. Consistent with the foregoing, multi-compartment, multi-phase capsules and encapsulation technology are herein contemplated to produce a delivery vehicle for delivering anti-inflammatory and anti-oxidant compounds to the body in a single dosage. A capsular format of the present invention may include the following composition:

| | |
|---|---|
| Receiving Chamber (218a): | |
| Rofecoxib | 25 mg |
| [12.5-25 mg/day] | |
| Receiving Chamber (218b): | |
| Vitamin E | 200 IU |
| [200-400 IU/day] | |

The incorporation of time-release coatings to varying the release rates of the active ingredients (e.g., Rofecoxib and Vitamin E) of the multi-compartment capsule 210 may be used to target key time intervals or events when the body may be most able to utilize the named active ingredients. Thus, the incorporation of time-release coatings in the encapsulation process when forming a multi-compartment capsule may be specifically designed to fit the needs and desires of numerous different users having similar conditions that are being targeted for treatment.

According to one presently preferred embodiment of the present invention, a therapeutically effective amount of Rofecoxib may be introduced into at least a portion of the receiving chamber 218a in the form of a solid and a therapeutically effective amount of Vitamin E may be introduced into at least a portion of the receiving chamber 218b of the primary capsule 211 in the form of a liquid.

In an alternative presently preferred embodiment of the present invention, therapeutically effective amounts of Rofecoxib and Vitamin E (active ingredients) may be introduced into receiving chambers of a multi-compartment capsule wherein Rofecoxib comprises a physical state (e.g., solid, liquid, gas or dispersion) different from the physical state of Vitamin E. As shown in FIG. 2, a therapeutically effective amount of Rofecoxib, in the form of a solid, may be introduced into receiving chamber 118 and 138 and a therapeutically effective amount of Vitamin E, in the form of a liquid, may be introduced into receiving chamber 128 of a multi-compartment capsule 110 of the present invention. The material forming the primary capsule shell 111 may be formulated in a manner allowing for immediate dissolution and release of the of the contents of receiving chamber 118. The material forming the secondary capsule shell 120 may be formulated in a manner allowing for either an immediate dissolution or a time-delayed dissolution and release of the contents of receiving chamber 128. The material forming the tertiary capsule shell 138 may be formulated in a manner allowing for time-delayed dissolution and release of the contents of receiving chamber 138. In this presently preferred embodiment of the present invention, a total daily dosage of Rofecoxib may be delivered as two separate dosages within a single oral dosage form. One presently preferred embodiment of the present invention thus makes for a more convenient dosage form.

Since the encapsulation process and multi-compartment, multi-phase capsule of the present invention are configured to apply to an anticipated treatment regime or medicinal design of a single dosage capsule, it will be readily appreciated that the introduction of one or more active ingredients into receiving chambers defined within a capsule is anticipated such that the various ingredients may be introduced in different receiving chambers to accommodate different treatment modalities. It is intended, therefore, that the examples provided herein be viewed as exemplary of the principles of the present invention, and not as restrictive to a particular structure or method for implementing those principles.

### EXAMPLE III

### Diphenhydramine Hydrochloride (solid) & Vitamin E (liquid)

As appreciated by those skilled in the art, allergic reactions are conditions wherein the immune system is stimulated to identify, segregate and dispose of exogenous chemicals which cannot be recognized by the body. Allergic reactions are often associated with the release of histamine, a chemical compound which produces changes in the permeability of blood vessels and the accumulation of other immune system cells. In some circumstances, it may be. desirable to modulate the amount of allergic response that is capable of being generated by the immune system.

Diphenhydramine belongs to a class of compounds which are given the functional name: histamine-1 (H₁) receptor antagonists. These compounds are more generally labeled as antihistamines. These antagonists are further divided according to their chemical structures. Diphenhydramine is an ethanolamine (aminoalkyl ether) derivative. Other chemical divisions may include, for example: ethylenediamine, propylamine, phenothiazine, piperazine. The ethanolamine division may include, for example: diphenhydramine, clemastine, dimenhydrinate, and doxylamine. As appreciated, the foregoing list is provided herein as exemplary and should not be considered exclusive or exhaustive.

Antihistamines block the interaction of the neurotransmitter, histamine, with H₁ receptors located in smooth muscle linings of the gastrointestinal tract, bronchial tract and large blood vessels. This blocking action may lead to marked relaxation in smooth muscle tone and is facilitative of numerous physiological processes including respiration.

The H₁ antagonists may also be divided according to their selectivity for central and peripheral H₁ receptors. A second-generation of H₁ has emerged in recent years. These agents have a greater selectivity for peripheral H₁ receptors. Second-generation H₁ receptor antagonists may include, for example: azelastine (ASTELIN®), cetirizine (ZYRTEC®), desloratadine (CLARINEX®), fexofenadine (ALLEGRA®) and loratadine (CLARITINn®).

Vitamin E, also named alpha-tocopherol, is a well-known scavenger of free-radicals in the body. Free-radical scavengers are sometimes referred to as anti-oxidants. This scavenging process is important for detoxifying the body of chemicals which are known to promote apoptosis, or programmed cell death. Apoptosis is a scientific description of cellular destruction. Although it is a popular anti-oxidant, Vitamin E is poorly soluble in water and thus can be administered only as a liquid-oil formulation or in an oil formulation enclosed in a soft elastic capsule.

In one presently preferred embodiment of the present invention, therapeutically effective amounts of Diphenhydramine and Vitamin E (active ingredients) may be introduced into receiving chambers of a multi-compartment capsule wherein at least two of the active ingredients have physical states (e.g., solid, liquid, gas or dispersion) that differ. Consistent with the foregoing, multi-compartment, multi-phase capsules and encapsulation technology are herein contemplated to produce a delivery vehicle for delivering anti-allergic and anti-oxidant compounds to the body in a single dosage. A capsular format of the present invention may include the following composition:

| | |
|---|---|
| Primary Capsule: | |
| Diphenhydramine HCl | 50 mg |
| [25-100 mg/day] | |
| Secondary Capsule: | |
| Vitamin E | 200 IU |
| [200-400 IU/day] | |

The incorporation of time-release coatings to varying the release rates of the active ingredients (e.g., Diphenhydramine and Vitamin E) in the primary and secondary capsules, respectively, of the multi-compartment capsule may be used to target key time intervals or events when the body may be most able to utilize the named active ingredients. Thus, the incorporation of time-release coatings in the encapsulation process when forming a multi-compartment capsule may be specifically designed to fit the needs and desires of numerous different users having similar conditions that are being targeted for treatment.

A therapeutically effective amount of Diphenhydramine may be introduced into at least a portion of the internal periphery of the receiving chambers of a primary capsule in the form of a solid and a therapeutically effective amount of Vitamin E may be introduced into at least a portion of a secondary capsule in the form of a liquid, if desired. Since the encapsulation process and multi-compartment, multi-phase capsule of the present invention are configured to apply to an anticipated treatment regime or medicinal design of a single dosage capsule, it will be readily appreciated that the introduction of one or more active ingredients into the receiving chambers of the primary and secondary capsules, respectively, is anticipated such that the various ingredients may be introduced in different receiving chambers to accommodate different treatment modalities. For example, a multi-compartment capsule may be formulated having Diphenhydramine introduced into the receiving chambers of the secondary capsule and Vitamin E may be introduced into the receiving chamber of the primary capsule. It is intended, therefore, that the examples provided herein be viewed as exemplary of the principles of the present invention, and not as restrictive to a particular structure or method for implementing those principles.

### EXAMPLE IV

### Celecoxib (solid) & Ibuprofen (liquid)

As appreciated by those skilled in the art, arthritis is an inflammatory condition typically affecting the synovia and cartilage of joints. It has been estimated that as many as one in three persons may experience symptoms associated with arthritis during their lifetime.

Anti-inflammatory agents may have many diverse therapeutic roles in the human body. Inflammation is the process undertaken by the body as it responds to an injury. A typical inflammatory response involves blood vessel dilation, increased blood flow to the site of injury, and influx of white blood cells to process and remove dead tissue. Inflammation can lead to pain and swelling at the site of injury. Medicaments used in modulating the inflammatory response may be divided into steroid and non-steroidal labels. The latter is more commonly identified as non-steroidal anti-inflammatory drugs (NSAIDs).

Celecoxib belongs to a class of NSAID compounds given the functional name cyclo-oxygenase-2 (“COX-2”) inhibitors. This class may include, for example: rofecoxib (VIOXX®), celecoxib (CELEBREX®), valdecoxib (BEXTRA®), etodolac (LODINE®) and meloxicam (MOBIC®). As appreciated, the foregoing list is provided herein as exemplary and should not be considered exclusive or exhaustive.

Celecoxib is believed to inhibit the action of COX-2, an enzyme involved in the production of prostaglandins in the human body. Prostaglandins serve many diverse roles, one of which is to stimulate an inflammation mechanism in immune responses. Recently, Celecoxib was labeled by the United States Food and Drug Administration (FDA) for use in the treatment of osteoarthritis, rheumatoid arthritis, acute pain, and primary dysmenorrhea.

Ibuprofen is another NSAID and is believed to function as a non-selective inhibitor of cyclo-oxygenase. Ibuprofen has been labeled by the FDA for use in the treatment of osteoarthritis, rheumatoid arthritis, relief of mild to moderate pain and primary dysmenorrhea. Ibuprofen belongs to a class of compounds called phenyl-a-methylacetic acids, which are derived from salicylic acid. Non-selective cyclo-oxygenase inhibitors may include, for example: ibuprofen (MOTRIN®), naproxen (NAPROSYN®), diclofenac (VOLTAREN®), flurbiprofen (ANSAID®), indomethacin (INDOCIN®), ketoprofen (ORUDIS®), ketorolac (TORADOL®), nabumetone (RELAFEN®), oxaprozin (DAYPRO®), piroxicam (FELDENE®) and sulindac (CLINORIL®). As appreciated, the foregoing list is provided herein as exemplary and should not be considered exclusive or exhaustive.

In one presently preferred embodiment of the present invention, therapeutically effective amounts of Celecoxib and Ibuprofen (active ingredients) may be introduced into receiving chambers of a multi-compartment capsule wherein Celecoxib comprises a physical state (e.g., solid, liquid, gas or dispersion) different from the physical state of Ibuprofen. As shown in FIGS. 3 and 4, a therapeutically effective amount of Celecoxib may be introduced into receiving chamber 218a and a therapeutically effective amount of Ibuprofen may be introduced into receiving chamber 218b of a multi-compartment capsule 210 of the present invention. Consistent with the foregoing, multi-compartment, multi-phase capsules and encapsulation technology are herein contemplated to produce a delivery vehicle for delivering anti-arthritic and anti-oxidant compounds to the body in a single dosage. A capsular format of the present invention may include the following composition:

Receiving Chamber (218a ):

| | |
|---|---|
| Receiving Chamber (218a): | |
| Celecoxib | 200 mg |
| [200-400 mg/day] | |
| Receiving Chamber (218b): | |
| Ibuprofen | 800 mg |
| [2400-3200 mg/day] | |

The incorporation of time-release coatings to varying the release rates of the active ingredients (e.g., Celecoxib and Ibuprofen) of the multi-compartment capsule 210 may be used to target key time intervals or events when the body may be most able to utilize the named active ingredients. Thus, the incorporation of time-release coatings in the encapsulation process when forming a multi-compartment capsule may be specifically designed to fit the needs and desires of numerous different users having similar conditions that are being targeted for treatment.

According to one presently preferred embodiment of the present invention, a therapeutically effective amount of Celecoxib may be introduced into at least a portion of the receiving chamber 218a in the form of a solid and a therapeutically effective amount of Ibuprofen may be introduced into at least a portion of the receiving chamber 218b of the primary capsule 211 in the form of a liquid.

In an alternative presently preferred embodiment of the present invention, therapeutically effective amounts of Celecoxib and Ibuprofen (active ingredients) may be introduced into receiving chambers of a multi-compartment capsule wherein Celecoxib comprises a physical state (e.g., solid, liquid, gas or dispersion) different from the physical state of Ibuprofen. As shown in FIG. 2, a therapeutically effective amount of Celecoxib, in the form of a solid, may be introduced into receiving chamber 128 and a therapeutically effective amount of Ibuprofen, in the form of a liquid, may be introduced into receiving chambers 118 and 138 of a multi-compartment capsule 110 of the present invention.

The material forming the primary capsule shell 111 may be formulated in a manner allowing for immediate dissolution and release of the of the contents of receiving chamber 118. The material forming the secondary capsule shell 120 may be formulated in a manner allowing for either an immediate dissolution or a time-delayed dissolution and release of the contents of receiving chamber 128. The material forming the tertiary capsule shell 138 may be formulated in a manner allowing for time-delayed dissolution and release of the contents of receiving chamber 138. In this presently preferred embodiment of the present invention, a total daily dosage of Ibuprofen may be delivered as two separate dosages within a single oral dosage form. One presently preferred embodiment of the present invention thus makes for a more convenient dosage form.

Since the encapsulation process and multi-compartment, multi-phase capsule of the present invention are configured to apply to an anticipated treatment regime or medicinal design of a single dosage capsule, it will be readily appreciated that the introduction of one or more active ingredients into receiving chambers defined within a capsule is anticipated such that the various ingredients may be introduced in different receiving chambers to accommodate different treatment modalities. It is intended, therefore, that the examples provided herein be viewed as exemplary of the principles of the present invention, and not as restrictive to a particular structure or method for implementing those principles.

From the above discussion, it will be appreciated that the present invention provides novel integrated capsule delivery apparatus and methods for delivering diverse physical states (e.g., solid, liquid, gas or dispersion) of a single active ingredient or medicament (e.g., pharmaceutical, biotechnical, nutraceutical, vitamin, dietary supplement, mineral or combination thereof), or a plurality of active ingredients or medicaments, in a single dosage capsular form, wherein at least two of the active ingredients or medicaments if different receiving chambers have physical states that differ. In preferred design, the encapsulation processes and multi-compartment capsular technology of the present invention may include various desirable properties such as, for example, controlling time-release of key active ingredients or medicaments, prolonging shelf-life of the active ingredients or medicaments, improving palatability, reducing overall production costs and reducing the number of capsules consumed by a patient or consumer as nutritional or therapeutic agents.

Unlike prior art multi-compartment capsular technology, the present invention provides novel integrated capsule delivery apparatus and methods for delivering a single dosage, multi-compartment capsule comprising a capsular base and cap configuration, wherein the size and shape of the cap, relative to its sealing relationship with the base, generally eliminates or substantially reduces any potential dead space volume within the internal periphery of the capsule, thereby functionally negating the opportunity for reaction between an air bubble and one or more active ingredients introduced into the capsule and, accordingly, improving stability of the capsular ingredient(s).

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. An encapsulation process for forming a multi-compartment capsule, said process comprising the steps of:
providing a primary capsule having a base and a cap;
providing a secondary capsule having a base and a cap;
introducing at least one ingredient having a first physical state into said secondary capsule;
positioning said cap of said secondary capsule in sealing relationship with said base;
introducing at least one ingredient having a second physical state into said primary capsule, wherein said first physical state of said ingredient of said secondary capsule is different from said second physical state of said ingredient of said primary capsule;
introducing said secondary capsule into said base of said primary capsule; and
positioning said cap of said primary capsule in sealing relationship with said base.

2. An encapsulation process as defined in claim 1, further comprising the step of reducing dead volume space within said primary capsule.

3. An encapsulation process as defined in claim 1, further comprising the step of introducing a filling material into said cap of said primary capsule to reduce dead volume space.

4. An encapsulation process as defined in claim 3, wherein said filling material is selected from the group consisting of gelatin, starch, casein, chitosan, soya bean protein, safflower protein, alginates, gellan gum, carrageenan, xanthan gum, phtalated gelatin, succinated gelatin, cellulosephtalate-acetate, polyvinylacetate, hydroxypropyl methyl cellulose, oleoresin, polyvinylacetate-phtalate, polymerisates of acrylic or methacrylic esters and combinations thereof.

5. An encapsulation process as defined in claim 1, wherein said cap of said primary capsule comprises a configuration sufficient for reducing dead volume space within the primary capsule.

6. An encapsulation process as defined in claim 1, wherein said ingredient introduced into said primary capsule is selected from the group consisting of a pharmaceutical, a biotechnical, a nutraceutical, a vitamin, a dietary supplement and a mineral.

7. An encapsulation process as defined in claim 1, wherein said physical state of said ingredient in said primary capsule is selected from the group consisting of a solid, a liquid, a gas and a dispersion.

8. An encapsulation process as defined in claim 7, wherein said solid is selected from the group consisting of a pill, a tablet, a capsule, a powder, granulation, flakes, a troche, a suppository, an ointment, a paste, an emulsion and a cream.

9. An encapsulation process as defined in claim 7, wherein said liquid is selected from the group consisting of a solution, a spirit, an elixir, a spray, a syrup and a fluid extract.

10. An encapsulation process as defined in claim 7, wherein said dispersion is selected from the group consisting of an aerosol, a suspension, an emulsion, a foam, a solid foam and a gel.

11. An encapsulation process as defined in claim 1, wherein said ingredient in said secondary capsule is selected from the group consisting of a pharmaceutical, a biotechnical, a nutraceutical, a vitamin, a dietary supplement and a mineral.

12. An encapsulation process as defined in claim 1, wherein said physical state of said ingredient in said secondary capsule is selected from the group consisting of a solid, a liquid, a gas and a dispersion.

13. An encapsulation process as defined in claim 12, wherein said solid is selected from the group consisting of a pill, a tablet, a capsule, a powder, granulation, flakes, a troche, a suppository, an ointment, a paste, an emulsion and a cream.

14. An encapsulation process as defined in claim 12, wherein said liquid is selected from the group consisting of a solution, a spirit, an elixir, a spray, a syrup and a fluid extract.

15. An encapsulation process as defined in claim 12, wherein said dispersion is selected from the group consisting of an aerosol, a suspension, an emulsion, a foam, a solid foam and a gel.

16. An encapsulation process as defined in claim 1, wherein said ingredient in said primary capsule comprises a pharmaceutical and said ingredient in said secondary capsule is selected from the group consisting of a pharmaceutical.

17. An encapsulation process as defined in claim 1, wherein said ingredient in said primary capsule comprises a pharmaceutical and said ingredient in said secondary capsule is selected from the group consisting of a biotechnical, a nutraceutical, a vitamin, a dietary supplement and a mineral.

18. An encapsulation process as defined in claim 1, wherein said ingredient introduced into said primary capsule is the same as said ingredient introduced into said secondary capsule.

19. An encapsulation process as defined in claim 1, wherein said primary capsule comprises a time-release coating.

20. An encapsulation process as defined in claim 1, wherein said secondary capsule comprises a time-release coating.

21. An encapsulation process as defined in claim 20, wherein said time-release coating of said secondary capsule is different from said time-release coating of said primary capsule.

22. An encapsulation process as defined in claim 1, further comprising the steps of:
providing a tertiary capsule having a base and a cap;
introducing at least one ingredient having a third physical state into said tertiary capsule;
positioning said cap of said secondary capsule in sealing relationship with said base; and
introducing said tertiary capsule into said base of said secondary capsule.

23. An encapsulation process as defined in claim 22, wherein said ingredient in said tertiary capsule is selected from the group consisting of a pharmaceutical, a biotechnical, a nutraceutical, a vitamin, a dietary supplement and a mineral.

24. An encapsulation process as defined in claim 23, wherein said ingredient in said tertiary capsule comprises a physical state selected from the group consisting of a solid, a liquid, a gas and a dispersion.

25. An encapsulation process as defined in claim 24, wherein said solid is selected from the group consisting of a pill, a tablet, a capsule, a powder, granulation, flakes, a troche, a suppository, an ointment, a paste, an emulsion and a cream.

26. An encapsulation process as defined in claim 24, wherein said liquid is selected from the group consisting of a solution, a spirit, an elixir, a spray, a syrup and a fluid extract.

27. An encapsulation process as defined in claim 24, wherein said dispersion is selected from the group consisting of an aerosol, a suspension, an emulsion, a foam, a solid foam and a gel.

28. An encapsulation process as defined in claim 22, wherein said tertiary capsule comprises a time-release coating.

29. An encapsulation process as defined in claim 1, wherein said primary capsule is formed of a material selected from the group consisting of gelatin, starch, casein, chitosan, soya bean protein, safflower protein, alginates, gellan gum, carrageenan, xanthan gum, phtalated gelatin, succinated gelatin, cellulosephtalate-acetate, polyvinylacetate, hydroxypropyl methyl cellulose, oleoresin, polymerisates of acrylic or mthacrylic esters, polyvinylacetate-phtalate and combinations thereof.

30. An encapsulation process as defined in claim 29, wherein said primary capsule further comprises a soft elastic capsule formed of a material selected from the group consisting of glycerin and sorbitol.

31. An encapsulation process as defined in claim 30, wherein said soft elastic capsule includes an antimicrobial selected from the group consisting of paraben and sorbic acid.

32. An encapsulation process as defined in claim 1, wherein said secondary capsule is formed of a material selected from the group consisting of gelatin, starch, casein, chitosan, soya bean protein, safflower protein, alginates, gellan gum, carrageenan, xanthan gum, phtalated gelatin, succinated gelatin, cellulosephtalate-acetate, polyvinylacetate, hydroxypropyl methyl cellulose, oleoresin, polymerisates of acrylic or mthacrylic esters, polyvinylacetate-phtalate and combinations thereof.

33. An encapsulation process as defined in claim 1, wherein said ingredient introduced in said primary capsule comprises a moisture content in the range of about 0% to 6% by weight.

34. A multi-compartment capsule as defined in claim 1, wherein said ingredient introduced in said secondary capsule comprises a moisture content in the range of about 0% to 6% by weight.

35. An encapsulation process as defined in claim 1, wherein said primary and secondary capsules contain at least one pharmaceutically acceptable lubricant in the range of about 0% to 10% by weight.

36. An encapsulation process as defined in claim 35, wherein said lubricant is selected from the group consisting of aluminiumstearate, calciumstearate, magnesiumstearate, tinstearate, talc, sodium lauryl sulfate, lecithins, mineral oils, stearic acid, silicones and combinations thereof.

37. An encapsulation process as defined in claim 1, wherein said primary and secondary capsules are formed having different colors.

38. An encapsulation process for forming a multi-compartment capsule, said process comprising the steps of:
providing a capsule comprising a cap, a base configured having a longitudinally extending body including a length and at least one dividing wall formed along said length of said extending body, said dividing wall adapted to form a first receiving chamber and a second receiving chamber;
introducing at least one ingredient having a first physical state into said second receiving chamber;
introducing at least one ingredient having a second physical state into said first receiving chamber, wherein said first physical state of said ingredient of said second receiving chamber being different from said second physical state of said ingredient of said first receiving chamber; and
positioning said cap in sealing relationship with said base.

39. An encapsulation process as defined in claim 38, further comprising the step of reducing dead volume space within said primary capsule.

40. An encapsulation process as defined in claim 38, further comprising the step of introducing a filling material into said cap to reduce dead volume space.

41. An encapsulation process as defined in claim 40, wherein said filling material is selected from the group consisting of gelatin, starch, casein, chitosan, soya bean protein, safflower protein, alginates, gellan gum, carrageenan, xanthan gum, phtalated gelatin, succinated gelatin, cellulosephtalate-acetate, polyvinylacetate, hydroxypropyl methyl cellulose, polyvinylacetate-phtalate, polymerisates of acrylic or methacrylic esters and combinations thereof.

42. An encapsulation process as defined in claim 38, wherein said cap comprises a configuration sufficient for reducing dead volume space within said capsule.

43. An encapsulation process as defined in claim 38, wherein said ingredient in said first receiving chamber is selected from the group consisting of a pharmaceutical, a biotechnical, a nutraceutical, a vitamin, a dietary supplement and a mineral.

44. An encapsulation process as defined in claim 38, wherein said physical state of said ingredient in said receiving chamber is selected from the group consisting of a solid, a liquid, a gas and a dispersion.

45. An encapsulation process as defined in claim 44, wherein said solid is selected from the group consisting of a pill, a tablet, a capsule, a powder, granulation, flakes, a troche, a suppository, an ointment, a paste, an emulsion and a cream.

46. An encapsulation process as defined in claim 44, wherein said liquid is selected from the group consisting of a solution, a spirit, an elixir, a spray, a syrup and a fluid extract.

47. An encapsulation process as defined in claim 44, wherein said dispersion is selected from the group consisting of an aerosol, a suspension, an emulsion, a foam, a solid foam and a gel.

48. An encapsulation process as defined in claim 38, wherein said ingredient in said second receiving chamber is selected from the group consisting of a pharmaceutical, a biotechnical, a nutraceutical, a vitamin, a dietary supplement and a mineral.

49. An encapsulation process as defined in claim 38, wherein said physical state of said ingredient in said second receiving chamber is selected from the group consisting of a solid, a liquid, a gas and a dispersion.

50. An encapsulation process as defined in claim 49, wherein said solid is selected from the group consisting of a pill, a tablet, a capsule, a powder, granulation, flakes, a troche, a suppository, an ointment, a paste, an emulsion and a cream.

51. An encapsulation process as defined in claim 49, wherein said liquid is selected from the group consisting of a solution, a spirit, an elixir, a spray, a syrup and a fluid extract.

52. An encapsulation process as defined in claim 49, wherein said dispersion is selected from the group consisting of an aerosol, a suspension, an emulsion, a foam, a solid foam and a gel.

53. An encapsulation process as defined in claim 38, wherein said ingredient in said first receiving chamber comprises a pharmaceutical and said ingredient in said second receiving chamber is selected from the group consisting of a pharmaceutical.

54. An encapsulation process as defined in claim 38, wherein said ingredient in said first receiving chamber comprises a pharmaceutical and said ingredient in said second receiving chamber is selected from the group consisting of a biotechnical, a nutraceutical, a vitamin, a dietary supplement and a mineral.

55. An encapsulation process as defined in claim 38, wherein said first receiving chamber comprises a time-release coating.

56. An encapsulation process as defined in claim 38, wherein said second receiving chamber comprises a time-release coating.

57. An encapsulation process as defined in claim 56, wherein said time-release coating of said second receiving chamber is different from said time-release coating of said first receiving chamber.

58. An encapsulation process as defined in claim 38, further comprising the steps of:
positioning a second dividing wall along said length of said extending body of said base, said second dividing wall adapted to form a third receiving chamber; and
introducing at least one ingredient having a physical state into said third receiving chamber.

59. An encapsulation process as defined in claim 58, wherein said ingredient introduced into said third receiving chamber is selected from the group consisting of a pharmaceutical, a biotechnical, a nutraceutical, a vitamin, a dietary supplement and a mineral.

60. An encapsulation process as defined in claim 58, wherein said physical state of said ingredient introduced into said third receiving chamber is selected from the group consisting of a solid, a liquid, a gas and a dispersion.

61. An encapsulation process as defined in claim 60, wherein said solid is selected from the group consisting of a pill, a tablet, a capsule, a powder, granulation, flakes, a troche, a suppository, an ointment, a paste, an emulsion and a cream.

62. An encapsulation process as defined in claim 60, wherein said liquid is selected from the group consisting of a solution, a spirit, an elixir, a spray, a syrup and a fluid extract.

63. An encapsulation process as defined in claim 60, wherein said dispersion is selected from the group consisting of an aerosol, a suspension, an emulsion, a foam, a solid foam and a gel.

64. An encapsulation process as defined in claim 58, wherein said second dividing wall comprises a time-release coating.

65. An encapsulation process as defined in claim 38, wherein said capsule is formed of a material selected from the group consisting of gelatin, starch, casein, chitosan, soya bean protein, safflower protein, alginates, gellan gum, carrageenan, xanthan gum, phtalated gelatin, succinated gelatin, cellulosephtalate-acetate, polyvinylacetate, hydroxypropyl methyl cellulose, oleoresin, polymerisates of acrylic or methacrylic esters, polyvinylacetate-phtalate and combinations thereof.

66. An encapsulation process as defined in claim 65, wherein said capsule further comprises a soft elastic capsule formed of a material selected from the group consisting of glycerin and sorbitol.

67. An encapsulation process as defined in claim 66, wherein said soft elastic capsule includes an antimicrobial selected from the group consisting of paraben and sorbic acid.

68. An encapsulation process as defined in claim 38, wherein said dividing wall of said capsule is formed of a material selected from the group consisting of gelatin, starch, casein, chitosan, soya bean protein, safflower protein, alginates, gellan gum, carrageenan, xanthan gum, phtalated gelatin, succinated gelatin, cellulosephtalate-acetate, polyvinylacetate, hydroxypropyl methyl cellulose, oleoresin, polymerisates of acrylic or methacrylic esters, polyvinylacetate-phtalate and combinations thereof.

69. An encapsulation process as defined in claim 38, wherein said ingredient introduced in said primary capsule comprises a moisture content in the range of about 0% to 6% by weight.

70. A multi-compartment capsule as defined in claim 38, wherein said ingredient introduced in said secondary capsule comprises a moisture content in the range of about 0% to 6% by weight.

71. An encapsulation process as defined in claim 38, wherein said primary and secondary capsules contain at least one pharmaceutically acceptable lubricant in the range of about 0% to 10% by weight.

72. An encapsulation process as defined in claim 71, wherein said lubricant is selected from the group consisting of aluminiumstearate, calciumstearate, magnesiumstearate, tinstearate, talc, sodium lauryl sulfate, lecithins, mineral oils, stearic acid, silicones and combinations thereof.

73. An encapsulation process as defined in claim 38, wherein said base and said cap of said capsule are formed having different colors.

74. An encapsulation process as defined in claim 38, further comprising the step of introducing two or more dividing walls adapted to form a plurality of receiving chambers into said base of said capsule.

75. An encapsulation process as defined in claim 74, further comprising the step of introducing a second capsule into one of said plurality of receiving chambers.

76. An encapsulation process as defined in claim 75, wherein said second capsule may comprise a multi-compartment capsule.
